SEQUENCE LISTING (8 sequences)

SEQ ID NO: 1 - DNA, 20 bp, Mus sp.
cctggctgttcccctatgaa

SEQ ID NO: 2 - DNA, 21 bp, Mus sp.
gagctggtgggagtgtcagtg

SEQ ID NO: 3 - DNA, 21 bp, Mus sp.
ctctggccctgcttattgttg

SEQ ID NO: 4 - DNA, 21 bp, Mus sp.
gagctggtgggagtgtcagtg

SEQ ID NO: 5 - DNA, 21 bp, Mus sp.
ctctggccctgcttattgttg

SEQ ID NO: 6 - DNA, 24 bp, Mus sp.
gaagacatttgggaaggactgact

SEQ ID NO: 7 - DNA, 21 bp, Mus sp.
gatgacgatatcgctgcgctg

SEQ ID NO: 8 - DNA, 21 bp, Mus sp.
gtacgaccagaggcatacagg

(19) United States
(12) Patent Application Publication
(10) Pub. No.: US 2009/0117046 A1
(43) Pub. Date: May 7, 2009

(54) METHODS FOR EVALUATING GENETIC SUSCEPTIBILITY AND THERAPY FOR CHRONIC INFLAMMATORY DISEASES

(75) Inventors: Yoram GRONER, Rehovot (IL); David Bettoun, Merion Station, PA (US)

Correspondence Address: WINSTON & STRAWN LLP;PATENT DEPARTMENT, 1700 K STREET, N.W., WASHINGTON, DC, 20006, US

(73) Assignee: YEDA RESEARCH AND DEVELOPMENT, LTD.

(21) Appl. No.: 12/352,460

(22) Filed: Jan. 12, 2009

Related U.S. Application Data
(62) Division of application No. 10/540,402, filed on Jun. 30, 2006.
(60) Provisional application No. 60/436,655, filed on Dec. 30, 2002.
(60) Provisional application No. 60/472,423, filed on May 22, 2003.

(51) Int. Cl. A61K 49/00; C12Q 1/68; A61P 43/00

(52) U.S. Cl. 424/9.2; 435/6

(57) ABSTRACT

The invention discloses a knockout mouse that is homozygous for a RUNX3 null allele, as a novel model for chronic inflammatory disease. The present invention provides methods of diagnosing and assessing predisposition to chronic inflammatory diseases. The present invention further provides methods for testing agents for effectiveness in treating and/or preventing diseases associated with chronic inflammatory or autoimmune conditions.

### FIELD OF THE INVENTION

The present invention provides methods for treating T cell-related inflammatory conditions. The invention further relates to methods of testing agents for effectiveness in treating and/or preventing chronic inflammatory diseases and to methods for assessing predisposition to chronic inflammatory diseases.

### BACKGROUND OF THE INVENTION

Inflammatory and immune reactions depend upon the recruitment and migration of circulating leukocytes to sites of injury or antigen exposure. Accumulation and activation of leukocytes result in the generation of numerous cytokines, growth factors, enzymes, and mediators, which participate in the further recruitment and activation of leukocytes, thereby augmenting and propagating the defense of the injured or antigen-exposed mammal.

Dendritic cells (DC) are sparsely distributed, migratory bone-marrow-derived cells that are specialized in uptake, processing and presentation of antigens to T cells (Banchereau et al., 1998). The DC compartment is defined by surface expression of major histocompatibility complex class II (MHC II) and the β2-integrin CD11c, which is found on all DC, except Langerhans cells (LC). The latter are a specialized class of DC, which reside in the epidermis and whose development is uniquely dependent on the cytokine transforming growth factor β (TGF-β).

At the immature state DC monitor the antigenic environment for the presence of microorganisms. Detection of damage or pathogen-associated molecular patterns (PAMPs) such as lipopolysaccharides (LPS) and double-stranded RNA by tissue-resident-DC, initiates maturation of DC and their migration to the lymph nodes. Maturation is associated with upregulation of MHC II molecules and co-stimulatory molecules such as CD80 and CD86 (Banchereau and Steinman, 1998). Mature DC are unrivaled in their potential to stimulate naïve T cells.

The mucosal surfaces of respiratory and intestinal tracts are constantly exposed to environmental antigens and it has been speculated that in order to prevent overt inflammation in lungs and intestine, activation of Antigen Presenting Cells (APC) at these sites might be continuously attenuated by immunosuppressive cytokines such as TGF-β (Nathan, 2002). Supporting this notion, absence of TGF-β was reported to result in lung inflammation (Nathan, 2002).

Numerous studies highlight the involvement of DC in the development of eosinophilic airway inflammation and asthma (Holt, 2000). More recent data have suggested an additional critical role in lung inflammation pathogenesis for a distinct subset of alveolar DC. These DC capture airborne antigens and maintain capacity to activate specific T cells long after antigen exposure (Julia et al., 2002). In the normal steady-state these cells comprise a minor fraction of the alveolar cell population, but they were reported to expand considerably in lungs with ongoing Th2 immune responses (Julia et al., 2002).

Chronic obstructive pulmonary diseases have a multifactorial etiology with an important component of genetic susceptibility, which modulates the individual's response towards environmental risk factors. The genetic background may influence the risk of disease for subjects exposed to environmental or occupational insults. Specifically, asthma and other respiratory diseases run in families indicating a strong genetic component. A genetic linkage between asthma and specific genes or markers is disclosed for example in U.S. Pat. No. 6,087,485 and in the references cited therein.

Chronic inflammatory disorders of the gastrointestinal tract are generally grouped under the heading of inflammatory bowel disease, although the disease can affect any part of the gastrointestinal tract from the esophagus to the large intestine. Inflammatory bowel disease is of unknown etiology, although psychological, immunologic, and genetic sources have been discussed as possible etiologic factors. The gastrointestinal inflammation associated with inflammatory bowel disease causes a range of symptoms of increasing severity and with a variety of intestinal and extraintestinal manifestations.

Experimentally induced animal models of inflammatory bowel disease are usually produced by exposure to toxic dietary substances, pharmacologic agents or other environmental chemicals, or by administration of materials derived from patients, or by manipulation of the animal's immune system (for review see: Beekan, W. L., Experimental inflammatory bowel disease, in: Kirsner, J. B., et al., eds.). One of the problems with experimentally-induced animal models for inflammatory bowel diseases is that the accompanying inflammation is very transient and cannot serve as a model of chronic ulcerative colitis.

Despite their limitations, the two most widely used models are the experimental colonic lesions produced by 2,4,6-trinitro-benzensulfonic acid (TNB) and carrageenan. Both models involve tissue destruction in the colon. Intrarectal administration of 5-30 mg of TNB in 0.25 ml of 50% ethanol in the rat produced dose-dependent colonic ulcers and inflammation which were maximal by gross and light microscopic examination at week, and by biochemical measurement of myeloperoxidase activity in the colon at 3-4 weeks (Morris, G. P., et al., Gastroenterology 96:795-803 (1989)). U.S. Pat. No. 5,214,066 discloses an animal model for inflammatory bowel disease including ulcerative colitis. Topical administration of sulfhydryl blockers such as N-ethylmaleimide and iodoacetamide in rodent colon induces chronic ulcerative colitis.

A widely used animal model for acute asthma is the ovalbumin (OVA)-sensitized mice in which intraperitoneal injections of OVA in adjuvant produces the symptoms of asthma (disclosed for example in U.S. Pat. No. 6,462,020). This mouse model of long-term repeated exposure to an allergen has been used to study the long-term effect of allergic diseases in the lung.

It is desirable to have animal models for chronic inflammatory diseases, including animals characterized by suppression of the expression of genes through genetic manipulation. These animal models would be very useful for identifying pharmaceutical agents that are able to treat or prevent these diseases. The present invention utilizes knockout mice that are homozygous for a RUNX3 null allele, previously described in Levanon et al. (The EMBO J., Vol 21 (13), pp. 3454-3463, 2002). Levanon et al disclosed that the knockout mice that are homozygous for a RUNX3 null allele exhibited certain neurological abnormalities associated with the development and survival of dorsal root ganglia proprioceptive neurons.

There is still an unmet need for methods and therapeutic agents for manipulating T cell-mediated reactions in patients in need thereof. The present invention provides methods for screening and identifying new therapeutic agents using novel models of inflammatory disease, and methods for diagnosis of genetic predisposition to such diseases, as well as cell therapy or gene therapy therefor.

### SUMMARY OF THE INVENTION

The present invention provides novel models for autoimmune or chronic inflammatory disease, particular chronic inflammatory diseases of the lung or gastrointestinal tract. The present invention further provides methods of screening putative drug candidates for therapeutic utility, using animals susceptible to chronic inflammatory disease or cells derived therefrom. The present invention further provides a new marker for genetic predisposition of individuals to development of such autoimmune or chronic inflammatory diseases. The genetic markers disclosed herein provides a new target for intervention using methods of cell therapy, gene therapy or antisense therapy, as appropriate in various clinical states.

The present invention is based in part on the unexpected observation that mice that are homozygous for a RUNX3 null allele or knockout mice (hereinafter RUNX3 KO mice) develop various inflammatory diseases, specifically eosinophilic lung inflammation and idiopathic inflammatory bowel disease. It was further discovered that the inflammatory state associated with these mice is characterized by a high proportion of mature dendritic cells (DC) versus immature DC, leading to increased potency to stimulate T cells.

Moreover, as disclosed herein below, these original findings prompted genetic association studies in human populations, providing indications for the association of variations in the RUNX3 gene, particularly in regulatory regions of the gene, with a late onset form of asthma. These observations, when taken together with the biological information pertaining to RUNX3 function, support the involvement of RUNX3 in at least some forms of human inflammatory disease.

In one aspect, the present invention relates to a method for inhibiting inflammation in a subject in need thereof, comprising contacting cells of the subject with an active agent that induces up-regulation of RUNX3 expression in the cells.

In one embodiment, the present invention relates to a method for inhibiting inflammation in a subject in need thereof, comprising contacting dendritic cells of the subject with an active agent that induces up-regulation of RUNX3 expression. Without wishing to be bound by any particular mechanism or theory of action, when the cells of the subject are DC, the agent may act by reducing the proportion of mature DC versus immature DC in said subject, thereby inhibiting inflammation.

The proportion of mature DC versus immature DC may be determined for example by evaluating the expression of specific markers associated with mature DC such as the T cell co-stimulatory molecules CD80, CD86, MHC class II and OX40L. Up-regulation of RUNX3 expression in DC may be achieved in vivo for example by using viral-based gene therapy methods known in the art.

Alternatively, DC may be obtained from the subject and the up-regulation of RUNX3 expression may be achieved in vitro for example by cell transfection, infection or any other means for introducing the active agent into the cells. According to embodiments wherein the cells are exposed to the active agent ex vivo, the transfected cells may be introduced back to the subject.

The present invention further comprises compositions for inhibiting T cell-mediated inflammation comprising as an active ingredient an agent that induces up-regulation of RUNX3 expression in cells. The compositions for inhibiting T cell-mediated inflammation are useful in situations where it is desirable to down-modulate an immune response, for example in a transplant patient (e.g., a recipient of an organ graft or bone marrow graft, etc.) or a subject suffering from an autoimmune disease including but not limited to systemic lupus erythematosus (SLE), rheumatoid arthritis (RA) and other forms of arthritis, multiple sclerosis (MS), ulcerative colitis, Crohn's disease, pancreatitis, diabetes, psoriasis, or other disorders associated with an abnormal immune response.

Suitable cell populations for modulation of RUNX3 expression are cells of the immune system, particularly thymocytes and dendritic cells.

According to one embodiment the present invention further comprises compositions for inhibiting T cell-mediated inflammation comprising as an active ingredient an agent that induces up-regulation of RUNX3 expression in DC.

The pharmaceutical compositions may contain in addition to the active ingredient conventional pharmaceutically acceptable carriers, diluents and excipients necessary to produce a physiologically acceptable and stable formulation.

The pharmaceutical compositions can be administered by any conventional and appropriate route including oral, parenteral, intravenous, intramuscular, intralesional, subcutaneous, transdermal, intrathecal, rectal or intranasal.

Prior to use as medicaments for preventing, alleviating or treating an individual in need thereof, the pharmaceutical compositions may be formulated in unit dosage. The selected dosage of active ingredient depends upon the desired therapeutic effect, the route of administration and the duration of treatment desired.

In another aspect, the present invention relates to a method of inhibiting the proliferation of T lymphocytes, comprising up-regulating the expression of RUNX3 in cells of an individual in need thereof. This method may be used in conditions in which inhibition of T lymphocytes is required such as in chronic inflammatory diseases, in T cell-mediated autoimmune diseases or in tissue transplantation.

According to one embodiment, the present invention relates to a method of inhibiting the proliferation of T lymphocytes, comprising up-regulating the expression of RUNX3 in the DC. This method may be used in conditions in which inhibition of T lymphocytes is required such as in chronic inflammatory diseases, in T cell-mediated autoimmune diseases or in tissue transplantation. Without wishing to be bound by any mechanism or theory of action, the upregulation of RUNX3 may act by inhibiting the maturation of the DC required for inducing T lymphocyte proliferation.

In another aspect, the present invention relates to a method of attenuating DC maturation, comprising contacting the DC with an active agent that up-regulates the expression of RUNX3 in the DC, thereby attenuating the maturation of the DC. Up-regulation of RUNX3 expression in DC may be achieved in vivo for example by using viral-based gene therapy methods known in the art. Alternatively, DC may be obtained from the subject and the up-regulation of RUNX3 expression may be achieved in vitro for example by cell transfection. The transfected cells may be introduced back to the subject.

This method of inhibiting DC maturation may be used in conditions in which inhibition of T cell-mediated inflammation is required such as in chronic inflammatory diseases, and T cell-mediated autoimmune diseases including but not limited to systemic lupus erythematosus (SLE), rheumatoid arthritis (RA) and other forms of arthritis, multiple sclerosis (MS), ulcerative colitis, Crohn's disease, pancreatitis, diabetes, psoriasis, or tissue transplantation.

In another aspect, the present invention relates to a method for enhancing T cell-mediated immune response, comprising contacting cells of an individual in need thereof with an active agent that down-regulates the expression of RUNX3 in the cells, thereby enhancing at least one T cell-mediated immune response. Down-regulation of RUNX3 expression in cells may be achieved for example by using anti-sense technology or inhibition of RUNX3 promoter activity.

According to one embodiment, the present invention relates to a method for enhancing at least one T cell-mediated immune response, comprising contacting DC from an individual in need thereof with an active agent that down-regulates the expression of RUNX3 in the DC, thereby enhancing the at least one T cell-mediated immune response. Down-regulation of RUNX3 expression in DC may be achieved for example by using anti-sense technology or inhibition of RUNX3 promoter activity. Without wishing to be bound by any mechanism or theory of action the down regulation of RUNX3 enhances the maturation of the DC, thereby enhancing at least one T cell-mediated immune response.

The present invention further comprises compositions for enhancing T cell-mediated immune response comprising as an active ingredient an agent that induces down-regulation of RUNX3 expression in cells of an individual in need thereof. The method and compositions for enhancing the T cell responses are useful in situations where it is desirable to upregulate an immune response. For example the ability of a subject to mount a response against a tumor in a tumor-bearing subject can be stimulated, or a response against a pathogen (e.g., a bacteria, a virus, such as HIV, fungus, parasite etc.) in a subject suffering from an infectious disease can be stimulated. Additionally, the methods can be used to enhance the efficacy of vaccination.

According to one embodiment the composition is brought into contact with cells of the immune system. According to one currently preferred embodiment the cells are selected from thymocytes and DC.

In another aspect, the present invention provides a method of testing the efficacy of a treatment for a chronic inflammatory disease comprising subjecting cells derived from RUNX3 KO mice to a putative treatment in vitro and determining the efficacy of said treatment. According to the invention, a test agent can be administered to cells derived from RUNX3 KO mice and the ability of the agent to ameliorate the symptoms exhibited by cells derived from RUNX3 KO in vitro can be scored as having effectiveness against said diseases. In a preferred embodiment, the in vitro testing is performed with DC obtained from the RUNX3 KO mouse.

In a preferred embodiment, the test agent can be scored as having effectiveness in reducing the proportion of mature DC versus immature DC in RUNX3 KO-derived DC. Reducing the proportion of mature DC versus immature DC may be determined for example by reduced potency to stimulate T cells, reduced expression of MHC class II as well as the T cell co-stimulatory molecules CD80, CD86 and OX40L, and increased responsiveness to TGF-β mediated maturation attenuation.

In another aspect, the present invention provides a method of testing the efficacy of a treatment for a chronic inflammatory disease comprising subjecting the RUNX3 KO mice to a putative treatment and determining the efficacy of said treatment, by measuring the severity of symptoms characteristic of said diseases exhibited by said knockout mouse, in comparison to the severity of symptoms exhibited by such knockout mice not exposed to the treatment.

The RUNX3 KO mouse exhibiting symptoms characteristic of pulmonary eosinophilia with a chronic inflammatory state as well as symptoms characteristic of idiopathic inflammatory bowel disease. According to the invention, a test agent can be administered to the RUNX3 KO mouse and the ability of the agent to ameliorate the pulmonary eosinophilia with a chronic inflammatory state or the idiopathic inflammatory bowel disease can be scored as having effectiveness against said diseases.

It is noted that the test agent may act to ameliorate the inflammatory symptoms in the RUNX3 KO mice by enhancing RUNX3 function in the RUNX3 KO mouse. In another embodiment, the test agent is not related to the function of RUNX3. Specifically, in one option the test agent may interfere with RUNX3-dependent TGF-β maturation attenuation of DC to ameliorate the inflammatory symptoms. In another option, the test agent may act to ameliorate the inflammatory symptoms in the RUNX3 KO mice via RUNX3-independent mechanisms.

The therapeutic agents to be tested in vivo may be administered in a variety of routes including but not limited to orally, topically, and parenterally e.g. subcutaneously, intraperitoneally, or intravenously.

The present invention also provides a method of predicting an increased risk for developing a chronic inflammatory disease in a subject comprising the steps of: (a) obtaining a test sample from a subject to be assessed; and (b) determining the expression of RUNX3 in said sample, wherein when the expression of RUNX3 in the test sample is diminished, the subject has an increased risk of susceptibility to a chronic inflammatory disease.

According to various preferred embodiments, the method of predicting a chronic inflammatory disease is used for predicting diseases associated with pulmonary eosinophilia with a chronic inflammatory state or idiopathic inflammatory bowel diseases.

In one embodiment, the prediction of an increased risk for a chronic inflammatory disease is performed by obtaining a sample from a subject, preferably a human subject. In a preferred embodiment, said sample is a blood sample, preferable a sample of peripheral blood mononuclear cells (PBMC).

The prediction of an increased risk for a chronic inflammatory disease may be performed by a number of methods. According to some embodiments the methods used determine the absence or presence of RUNX3 mRNA or expression product in patient cells. For example, detection may utilize western blot, RT-PCR, in situ hybridization, Northern blot or immunohistochemistry.

According to another aspect the invention provides kits for diagnosis of genetic susceptibility to a chronic inflammatory disease comprising at least one probe capable of determining at least one genotype associated with the RUNX3 locus, or the expression of the gene product encoded by this locus.

These and further embodiments will be apparent from the detailed description and examples that follow.

### BRIEF DESCRIPTION OF THE DRAWINGS

FIG. 1 is a fluorescence activated cell sorter (FACS) dot plot analysis obtained by gating the most mature HSA^{Low}TCR^{High} T lymphocytes of RUNX3 wild type (wt) and RUNX3 knockout (KO) mice.

FIG. 2 demonstrates the total amount of IL-5 and percentage of eosinophils in bronchoalveolar lavage fluid (BALF) of RUNX3 wild type (wt) and knockout (KO) mice.

FIG. 3 demonstrates eosinophil infiltration, mucus hypersecretion and signs of airway remodeling in lungs of RUNX3 KO mice. (A) Normal lungs in the WT mice showing airways and blood vessels surrounded by alveoli (x20, HE), (a/w: airway; b/v: blood vessel). (B) Lungs in the KO mice. The arrows denote infiltrating inflammatory cells, predominantly eosinophils, which accumulate in the interstitium around blood vessels and airways. A mild hypercellularity of the alveoli and alveolar septae denoted by arrowhead is seen in the low right hand side of the field. (X20, HE). (C) A thick eosinophilic perivascular cuff in a KO mouse (x40, HE). (D) High power view of an area in C. Eosinophils have eosinophilic cytoplasmic granules and lobed nuclei. The perivascular infiltrate is purely eosinophilic (x100, HE). The insert at the right hand corner depicts high power (x100) phenol red and DAPI stained eosinophils. Phenol red stains the cytoplasm bright red and the lobed nucleus stained with DAPI is easily identified. (E and F) WT and KO lungs stained with periodic acid Schiff (PAS), which stains mucus in purple. (E) PAS positive material is not present in WT (PAS x40). (F) In the KO, wispy PAS-positive material is observed in the cytoplasm of epithelial cells lining a small caliber airway. Arrows point to clusters of infiltrating inflammatory cells (PAS x40). (G) WT lung stained with Masson's trichrome (MT) which stains collagen fibers in blue. Small amount of collagen is present in the interstitium surrounding blood vessels and airways. Collagen fibers are concentrated around blood vessels and in the region where blood vessels and airways are in apposition (MT x20). (H) KO lung stained with MT. Collagen fibers are deposited in a disorganized manner among clusters of infiltrating inflammatory cells indicating airway remodeling (MT x20). (I and J) RUNX3 is highly expressed in activated DC/Macrophages from BAL of OVA challenged WT mice. Experimental acute asthma was induced in WT Balb/C mice. BAL cells were obtained and immunostained with anti Runx3 antibodies. (I) Conjugates of DC and T cells in which only DC show nuclear staining of Runx3 (brown), whereas the T cells are stained only by the hematoxylin counter-stain (blue). (J) Eosinophils (E) and neutrophils (N) are not stained with anti Runx3 antibodies.

FIG. 4 demonstrates that the F4/80/CD11c⁺/CD11b⁺/OX40L subset of alveolar DC is significantly elevated in BAL of RUNX3 KO mice. BAL of KO mice and WT littermates were obtained. Lavage fluid cells were stained with anti CD11c, anti CD11b, anti F4/80, anti OX40L and anti MHC II and analyzed by FACS. (A) Most of CD11c⁺ alveolar cells also expressed F4/80. (B) KO CD11c+/CD11b⁺ subset was elevated (from 3%±1 to 15%±5 n=4; p=0.04). (C and D) KO cells also express higher MHC II and OX40L compared to WT. OX40L is also elevated in the KO CD11c⁺/CD11b⁺ subset as shown by the histogram on the right.

FIG. 5 demonstrates that RUNX3 expression is induced upon maturation of BMDC. (A) Analysis of RUNX3 expression in sorted WT DC. Day 11 BMDC treated with LPS (1 μg/ml), stained with anti CD11c and anti MHC II and analyzed by FACS. DC were gated as high forward scatter cells (R1) and sorted into CD11c+mature, MHC II high (R2) and immature, MHC II low (R3), respectively. (B) Expression of RUNX3 in spontaneously matured WT BMDC. Western blot analysis of proteins from immature (7 days) and matured (14 days) cultured WT BMDC using anti Runx3 Ab. Note the similar intensity of the 85 kDa non-specific protein band in immature and mature BMDC. (C) Immunostaining of Runx3 in FACS sorted mature BMDC. Four thousand cells of either R2 or R3 were collected onto slides and immunostained with anti Runx3 Ab. (D) In spleen, RUNX3 expression is confined to the mature periarteriolar lymphoid sheath DC. Cryosections of spleens derived from CX₃CR1^{+/GFP} mice 6 h after injection of LPS (80 μg), stained with anti GFP and anti Runx3 Ab. Upper panels are at low magnification showing absence of RUNX3 expression (red nuclear staining) in marginal zone immature DC (green GFP positive cytoplasmic staining). Lower panels are at higher magnification showing RUNX3 expression in mature DC located in the periarteriolar lymphoid sheaths. The white pulp central artery is denoted by a white asterisk

FIG. 6 demonstrates that RUNX3 KO DC display enhanced maturation and increased ability to stimulate T-cell proliferation. Splenic DC of RUNX3 KO and WT mice were isolated, cultured overnight without or with LPS (1 μg/ml), stained and analyzed by flow cytometry (A-C). (A) CD11c⁺/CD19KO or WT DC were gated (R2). (D and C) Expression of MHC II and CD86, respectively in untreated (dotted line) and LPS-treated (solid line) WT and KO DC was measured. (D) Splenic DC were cultured in a suboptimal concentration of LPS (100 ng/ml) and analyzed for MHC II. Maturation was observed only in KO DC. (E) Syngeneic oxidative mitogenesis (left panel): Increasing number of WT and KO DC were incubated for 24 h with 3×10⁵ purified sodium periodate treated CD4⁺ T cells of the same mouse. MLR (right panel): DC were incubated for 64 h with 1×10⁵ purified CD4⁺ T cells from each of the three WT strains C57/BL, BALB/C and SJL. [³H]thymidine incorporation was determined as previously described (Woolf et al 2003). Results of oxidative mitogenesis are presented as cpm per CD11c⁺ DC (determined by FACS analysis). One experiment out of two with similar results is shown. MLR data represent the average at each point of [³H]thymidine incorporated by T cells of the three WT mouse strains. At the lower DC/T-cell ratio in the oxidative mitogenesis assay only the KO DC induced T cell proliferation.

FIG. 7 demonstrates the enhanced spontaneous maturation of RUNX3 KO BMDC Day 11 BMDC from cultures not treated with LPS were gated as high forward scatter/CD11c⁺ cells (R2) and assessed for expression of CD80 and MHC II. Solid lines, RUNX3 KO; dotted line, WT littermates.

FIG. 8 demonstrates the loss of TGF-β-mediated functions in RUNX3 KO DC compartment. (A) Epidermal sheaths were prepared and stained with PE conjugated anti MHC II Ab to detect Langerhans cells (LC). LC are seen in the WT preparation, but were absent in the KO. Upper and lower panels depict low and high magnifications, respectively. (B) Single cell suspension derived from epidermal sheaths of KO and WT mice, stained with MHC II and CD3 Ab and analyzed by FACS. T cell populations (R3) were similar in WT and KO, whereas LC (R2) are absent in the KO mice. (C) RUNX3 KO and WT BMDC were incubated with GM-CSF and without or with 10 ng/ml TGF-β. Day 7 cells (10⁶ cells/ml) were cultured overnight with 1 μg/ml LPS to induce maturation, collected and stained with anti CD11c and anti MHC II antibodies. Dendritic cells from KO or WT were gated as CD11c⁺ cells (R1) and assessed for expression of MHC II. TGF-β inhibited a significant part of the LPS-induced maturation reflected in an increase of MHC II^{low} cells, whereas maturation of KO BMDC was not affected by TGF-β.

FIG. 9 demonstrates that TGF-β dependent IgA class switching in cultured RUNX3 KO splenocytes is abrogated. (A) Splenocytes were cultured in the presence of TGF-β and LPS to induce IgA class switching. At day 4 RNA was prepared and analyzed by RT-PCR. IgA germline (IgA GL) and IgA post switch (ps IgA) transcripts were detected only in WT splenocytes, but not in the KO. Levels of IgM mRNA in WT and KO were similar. (B) Aliquots of supernatant from cultured splenocytes were removed on culture days 0, 3, 7, and 8 and levels of IgA, IgM and IgG were determined by ELISA. IgA production was detected in days 7 and 8, but only in WT splenocytes. Production of IgM and IgG was similar. (C) BAL from KO and WT mice were analyzed for IgA levels by ELISA (n=4, p=0.001). ELISA results are presented as the optical density readouts of the machine.

FIG. 10 shows the altered expression of β2-integrins in RUNX3 KO mice. (A) WT and RUNX3 KO splenic DC analyzed by FACS using anti CD11c, anti CD11b and anti CD11a antibodies. Cells were gated as high forward scatter/CD11c^{high} population (R1) and assessed for expression of the three β2-integrins. Solid and dotted lines represent RUNX3 KO and WT littermates, respectively. CD11a and CD11b were elevated in the KO compared to WT, whereas CD11c decreased. Expression of the β2-integrins common βchain (CD18) in WT and KO DC was similar. (B) Normal expression of β2-integrins in RUNX3 KO neutrophils. Peripheral blood leukocytes (PBL) of RUNX3 KO and WT mice stained with anti Gr-1, CD11b, and CD11a antibodies and neutrophils gated as high side scatter/CD11b⁺/Gr-1⁺ cells (R2). Expression of CD11b and CD11a in WT (dotted line) and KO (solid line) was monitored. Peritoneal lavage neutrophils were obtained following induction of peritonitis and analyzed for Runx3 by Western blots in parallel with proteins of WT thymus. Blots were reacted with anti Runx3 and anti I-□B Ab. (C) The CD8⁺ population of splenic DC is elevated in RUNX3 KO mice. Splenic DC of RUNX3 KO and WT littermate were reacted with anti CD11c, anti CD11b and anti-CD8α antibodies. High forward scatter/CD11c+ population (R1) of DC was gated and assessed for CD11b and CD8α. Percentage of CD8⁺/CD11b DC in the KO was elevated and that of CD8/CD11b⁺ reduced as compared to WT.

FIG. 11 demonstrates that RUNX3 KO mice exhibit inflammatory cellular infiltration in the cecum, colon and rectum.

FIG. 12 demonstrates pronounced hyperplasia of the glandular mucosa of the stomach of RUNX3 KO mice.

FIG. 13 demonstrates that inflammatory infiltrate is present in the proximal duodenum where it is associated with severe avillous hyperplasia.

### DETAILED DESCRIPTION OF THE INVENTION

in order that this invention may be better understood, the following terms and definitions are herein provided.

The term “Chronic Obstructive Pulmonary Disease” refers to a chronic disease which is characterized by airflow limitation (i.e., airflow obstruction or narrowing) due to constriction of airway smooth muscle, edema and hyper-secretion of mucous leading to increased work in breathing, dyspnea, hypoxemia and hypercapnia.

The term “non-atopic asthma” refers to a reversible airflow limitation in the absence of allergies.

The term “atopic asthma” refers to an airflow limitation in the presence of allergies characterized by a predisposition to raise an IgE antibody response to common environmental antigens.

The term “RUNX3” refers to the runt-related transcription factor 3 gene which is localized on human chromosome 1p36.1 and on mouse chromosome 4. RUNX3 belongs to a family of transcription factors whose members contain a highly conserved region designated the ‘runt domain’ found in the Drosophila gene Runt. The runt domain mediates the binding of Runx3 protein to DNA as well as protein-protein interaction with other proteins.

The term “null allele” refers to an allele in which the wild-type copy of the gene undergoes targeted disruption so as to prevent expression of that gene in the cell.

The term “targeted disruption” refers to the site-specific interruption of a native DNA sequence so as to prevent expression of that gene in the cell as compared to the wild-type copy of the gene. The interruption may be caused by deletions, insertions or modifications to the gene, or any combination thereof.

The term “knock-out” refers to partial or complete suppression of the expression of an endogenous gene. This is generally accomplished by deleting a portion of the gene or by replacing a portion with a second sequence, but may also be caused by other modifications to the gene such as the introduction of stop codons, the mutation of critical amino acids, the removal of an intron junction, etc.

The term “homozygote knock-out” refers to a transgenic mammal with a knock-out (KO) construct on both members of a chromosome pair in all of its genome-containing cells.

The term “putative treatment” refers to any therapeutically active substance which is delivered to a living organism to produce a desired, usually beneficial effect. In general, this includes therapeutic agents in all of the major therapeutic areas, also including proteins, peptides, oligonucleotides, and carbohydrates as well as inorganic ions, such as for example calcium ion, lanthanum ion, potassium ion, magnesium ion, phosphate ion, lithium ion, selenium ion or chloride ion.

The terms “efficacy of said treatment” refers to changes in the phenotype of the RUNX3 KO mouse or changes in the phenotype of cells derived from the RUNX3 KO mouse. The changes can be either subjective or objective and can relate to features such as symptoms or signs of the disease or biochemical markers associated with the disease.

The term “mature dendritic cells” refers to dendritic cells (DC) having accelerated potency to stimulate T cells, increased expression of MHC class II as well as the T cell co-stimulatory molecules CD80, CD86 and OX40L.

The term “immature dendritic cells” refers to DC having low potency to stimulate T cells, and reduced expression of the co-stimulatory molecules CD80, CD86 and OX40L. The immature state of DC is mediated by TGF-β maturation attenuation.

Eosinophilic lung inflammation is a chronic obstructive pulmonary disease characterized by airflow limitation, such as chronic asthma. An eosinophilic lung inflammation-related disease refers to an acute pulmonary eosinophilia such as acute bronchial asthma. Other diseases associated with pulmonary activated eosinophils are for example transient pulmonary eosinophilic infiltrates (Loffler's syndrome), hypersensitivity pneumonia, allergic bronchopulmonary aspergillosis, tropical eosinophilia, and chronic eosinophilia pneumonia Inflammatory bowel diseases are for example Crohn's disease or ulcerative colitis.

As used herein, the terms “asthma” and “bronchial asthma” refer to a condition of the lungs in which there is widespread narrowing of lower airways. “Atopic asthma” and “allergic asthma” refer to asthma that is a manifestation of an IgE-mediated hypersensitivity reaction in the lower airways, including, e.g., moderate or severe chronic asthma, such as conditions requiring the frequent or constant use of inhaled or systemic steroids to control the asthma symptoms. A preferred indication is allergic asthma.

Crohn's Disease is an inflammatory bowel disease in which areas of the intestinal tract become inflamed causing sloughing and, in some instances, ulcers. While many other inflammatory bowel diseases cause inflammation of the intestinal lining, Crohn's affects all layers of the intestine, not just the surface. The most common symptoms include diarrhea and intense stomach pain.

Ulcerative colitis is an inflammatory bowel disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. Ulcerative colitis may also be called colitis or proctitis.

New Models for Diagnosis and Treatment of Inflammatory Disease

The methods of the invention employ the RUNX3 KO mouse, which was surprisingly found to develop a condition with symptoms of eosinophilic lung inflammation and an inflammatory bowel disease. The RUNX3 KO mouse may therefore serve as an animal model for these diseases. The symptoms of pulmonary eosinophilia appearing in the RUNX3 KO mouse include the following: heavy breathing, an increase in the CD4⁺ subset and a decrease in the CD8⁺ cytotoxic T cells (CTL) subset in thymus, spleen and blood, eosinophilic infiltration in the lung associated with increased number of lymphocytes, CD 11c+ CDs/macrophages and specifically the highly T-cell stimulatory CD11c+ subset of DCs/macrophages, increased total cell numbers in BALF (bronchoalveolar lavage fluid) and a significantly increased level of IL-5. Specifically, eosinophilic lung inflammation is commonly observed in atopic and non-atopic asthma and IL-5 production by activated CD4 T cells is enhanced in both atopic and non-atopic patients as compared to normal control subjects. The symptoms of inflammatory bowel disease appearing in the RUNX3 KO mouse include typhlocolitis, gastric mucosal hyperplasia/proliferative gastritis and proximal duodenitis.

The inventors of the present invention further discovered that DC derived from the RUNX3 KO mouse exhibit abolished response to TGF-β and the resulting unrestrained maturation of DC associated with an increase in the unique subset of alveolar DC that are F4/80⁺/CD11c⁺/CD11b⁺. This subset of DC, which is barely detectable in lungs of WT mice, was recently identified as a potent subset of APC, possessing a sustained allergen presentation capacity (Julia et al., 2002). Significantly, RUNX3 KO alveolar DC also expressed higher levels of the co-stimulatory molecule OX40L, which was shown to play a crucial role in the development of allergic inflammation in mice (Akbari et al., 2003). Moreover, not only are RUNX3 KO DC resistant to TGF-β mediated maturation attenuation, they also over-respond to various maturation inducing reagents including low levels of LPS, TNFα and anti CD40, resulting in accelerated maturation. The mature KO DC appeared highly potent displaying increased expression of MHC class II as well as the T cell co-stimulatory molecules CD80, CD86 and OX40L. Indeed, when tested in syngeneic and allogeneic mixed leukocytes reactions RUNX3 KO DC displayed significantly higher potency to stimulate T cells as compared to WT DC. Together, these occurrences are likely to cause overreaction of the KO DC population to innocuous airborne antigens resulting in elevation of highly potent alveolar DC that in turn activate T cells, culminating in enhanced recruitment of eosinophils to the lungs of KO mice.

The accelerated maturation of RUNX3 KO DC was also associated with aberrant expression of β2-integrins even though the expression of the common CD18 β-chain was unchanged. Intriguingly, while the expression of CD11c significantly decreased in the KO, the expression of CD11b and CD11a increased. This opposite effects on the expression of the β2-integrins α chains could have resulted from the bi-functional nature of Runx3, which can act both as an activator or a repressor of target gene transcription, through recruitment of the co-repressor Gro/TLE (Levanon et al., 1998).

The data presented here provide evidence for the importance of Runx3 function as a component of the TGF-β signaling cascade in DC development. When Runx3 is lost, epidermal LC are absent and KO DC display accelerated maturation due to lack of responsiveness to TGF-β and over-responsiveness to maturation inducing stimuli. The accelerated maturation/migration of the KO DC is associated with aberrant expression of β2-integrins, increased potency to activate T cells and with population imbalance in lungs and spleen. In the lung of the KO mice, a unique subset of alveolar DC is increased. The accumulation of these DC might reflect an over-response to otherwise innocuous airborne antigens and result in activation of T cells, which elicit enhanced recruitment of eosinophils to the lungs, leading to inflammation, mucus hypersecretion and airway remodeling in RUNX3 KO mice.

In one aspect, the present invention relates to a method of inhibiting inflammation in a subject in need thereof, comprising contacting DC of the subject with an active agent that induces up-regulation of RUNX3 expression in the DC, thereby reducing the proportion of mature DC versus immature DC in said subject, thereby inhibiting inflammation.

The proportion of mature DC versus immature DC may be determined for example by determining the expression of specific markers associated with mature DC such as the T cell co-stimulatory molecules CD80, CD86 and OX40L. Alternatively, the proportion of mature DC versus immature DC may be determined by the ability of the DC to induce T cell proliferation.

Up-regulation of RUNX3 expression in DC may be achieved in vivo for example by using virus-mediated gene-delivery systems e.g. a viral vector to deliver the DNA molecule encoding Runx3 to the target DC. The preferred vector to deliver the DNA molecule is a virus that has been genetically altered to carry the DNA encoding Runx3. The term “active agent” as used herein describes for example a DNA viral vector encoding the Runx3 protein to be inserted within the DC to induce over expression of RUNX3 in the DC. Alternatively, the active agent may be a DNA viral vector encoding a promoter activator which is capable of inducing up-regulation of RUNX3 expression in the DC.

Besides virus-mediated gene-delivery systems, there are several non-viral options for gene delivery which may be used. This includes a direct introduction of therapeutic DNA into the target cells. This approach is limited in its application because it can be used only with certain tissues and requires large amounts of DNA. Another non-viral approach involves the creation of an artificial lipid sphere with an aqueous core. This liposome, which carries the therapeutic DNA, is capable of passing the DNA through the target cell's membrane. Therapeutic DNA also can get inside target cells by chemically linking the DNA to a molecule that will bind to special cell receptors. Once bound to these receptors, the therapeutic DNA constructs are engulfed by the cell membrane and passed into the interior of the target cell. This delivery system tends to be less effective than other options.

In another option, the DC may be obtained from the subject and the up-regulation of RUNX3 expression may be achieved in vitro for example by inserting a DNA vector encoding Runx3 to the DC. Thus, a DNA vector encoding the Runx3 protein may be inserted within the DC using cell transfection procedures known in the art to induce over expression of RUNX3 in the DC. The transfected cells over-expressing RUNX3 may be introduced back to the subject.

In another aspect, the present invention relates to a method for enhancing the maturation of DC, comprising contacting the DC with an active agent that down-regulates the expression of RUNX3 in the DC, thereby enhancing the maturation of the DC.

Down-regulation of RUNX3 expression in DC may be achieved for example by using anti-sense technology to target the RNA molecules encoding Runx3. Antisense therapy employs modified stands of DNA that can bind to specific RNA sequences, such as the RNA molecules encoding RUNX3. When the modified DNA strands bind to the targeted RNA, the RNA can no longer be translated into protein.

In another aspect, the present invention relates to the development of drugs for the treatment of chronic inflammatory diseases such as eosinophilic lung inflammation-related diseases or inflammatory bowel diseases. In one embodiment, the drug screening identifies agents that provide a replacement or enhancement for Runx3 function in affected cells. Conversely, agents that reverse the Runx3 function may stimulate bronchial reactivity. Of particular interest are screening assays for agents that have a low toxicity for human cells.

In one embodiment, the present invention provides a method of testing the efficacy of a treatment for chronic inflammatory diseases such as chronic obstructive pulmonary diseases, eosinophilic lung inflammation-related diseases or inflammatory bowel diseases, comprising subjecting a RUNX3 KO mouse to a putative treatment and determining the efficacy of said treatment, said RUNX3 KO mouse exhibiting symptoms characteristic of eosinophilic lung inflammation, chronic obstructive pulmonary diseases and inflammatory bowel diseases. According to the invention, a test agent can be administered to the RUNX3 KO mouse and the ability of the agent to ameliorate the eosinophilic lung inflammation, the chronic obstructive pulmonary disease or the inflammatory bowel disease can be scored as having effectiveness against said diseases.

In another embodiment, the present invention provides a method of testing the efficacy of a treatment for a chronic inflammatory disease comprising subjecting cells derived from RUNX3 KO mouse to a putative treatment in vitro and determining the efficacy of said treatment. According to the invention, a test agent can be administered to cells derived from RUNX3 KO mouse and the ability of the agent to ameliorate the symptoms exhibited by cells derived from RUNX3 KO in vitro can be scored as having effectiveness against said diseases. In a preferred embodiment, the in vitro testing is performed with DC obtained from the RUNX3 KO mouse.

In a preferred embodiment, the test agent can be scored as having effectiveness in reducing the proportion of mature DC versus immature DC in RUNX3 KO-derived DC. Reducing the proportion of mature DC versus immature DC may be determined for example by reduced potency to stimulate T cells, reduced expression of MHC class II as well as the T cell co-stimulatory molecules CD80, CD86 and OX40L, and increased responsiveness to TGF-β mediated maturation attenuation.

The term “agent” as used herein describes any molecule, e.g. protein or pharmaceutical, with the capability of altering or mimicking the physiological function of Runx3. Generally, a plurality of assays are run in parallel with different agent concentrations to obtain a differential response to the various concentrations. Typically, one of these concentrations serves as a negative control, i.e. at zero concentration or below the level of detection.

The compounds having the desired pharmacological activity may be administered in a physiologically acceptable carrier to a host for treatment. The compounds may also be used to enhance Runx3 function. The pharmaceutical compositions can be administered by any conventional and appropriate route including oral, parenteral, intravenous, intramuscular, intralesional, subcutaneous, transdermal, intrathecal, rectal or intranasal. In addition, the therapeutic agent comprising a nucleic acid may be administered where appropriate by viral infection, or other vectors suitable for gene therapy and the like. Inhaled treatments are of particular interest. Depending upon the manner of introduction, the compounds may be formulated in a variety of ways. The concentration of therapeutically active compound in the formulation may vary as required from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The pharmaceutical compositions may contain in addition to the active ingredient conventional pharmaceutically acceptable carriers, diluents and excipients necessary to produce a physiologically acceptable and stable formulation.

Prior to their use as medicaments for preventing, alleviating or treating an individual in need thereof, the pharmaceutical compositions will be formulated in unit dosage. The selected dosage of active ingredient depends upon the desired therapeutic effect, the route of administration and the duration of treatment desired.

In another embodiment of this invention, the RUNX3 KO mouse model may be exposed to various agents, such as environmental agents, potential toxins, and cigarette smoke, in order to study the potential effect of those agents on the bronchial reactivity. Such a model may also be used to assess the ability of various therapies and treatments to avoid or lessen the effects, if any, of such toxins and agents.

In yet another embodiment, the present invention relates to the development of drugs not related to the function of Runx3, for the treatment of chronic inflammatory diseases such as chronic obstructive pulmonary diseases, pulmonary eosinophilia-related diseases or inflammatory bowel diseases. Drugs currently used to treat asthma include beta 2-agonists, glucocorticoids, theophylline, cromones, and anticholinergic agents. For acute, severe asthma, the inhaled beta 2-agonists are the most effective bronchodilators. Short-acting forms give rapid relief; long-acting agents provide sustained relief and help nocturnal asthma. First-line therapy for chronic asthma is inhaled glucocorticoids, the only currently available agents that reduce airway inflammation. Theophylline is a bronchodilator that is useful for severe and nocturnal asthma, but recent studies suggest that it may also have an immunomodulatory effect. Cromones work best for patients who have mild asthma: they have few adverse effects, but their activity is brief, so they must be given frequently. Cysteinyl leukotrienes are important mediators of asthma, and inhibition of their effects may represent a potential breakthrough in the therapy of allergic rhinitis and asthma.

In another embodiment, the present invention provides a method of predicting an increased risk for developing a chronic inflammatory disease such as a pulmonary eosinophilia-related disease, a chronic obstructive pulmonary disease or an inflammatory bowel disease in a subject comprising the steps of: (a) obtaining a test sample from an individual to be assessed; and (b) determining the expression of RUNX3 in said sample, wherein when the expression of RUNX3 in the test sample is diminished, the individual has an increased risk of susceptibility to said chronic inflammatory disease.

In one embodiment, the prediction of an increased risk for a chronic inflammatory disease is performed by obtaining a sample from a subject. In a preferred embodiment, said sample is a blood sample, preferable a sample of peripheral blood mononuclear cells (PBMC). The expression of RUNX3 in said sample is analyzed, wherein when the expression of RUNX3 in the test sample is diminished, the individual has an increased risk of susceptibility to a chronic obstructive pulmonary disease or a inflammatory bowel disease.

The expression of RUNX3 may be determined by any suitable method well known in the art. In one embodiment, Northern blot analysis may be used to detect RUNX3 mRNA as an indirect measure of the Runx3 protein using a probe that is complementary to at least a portion of the RUNX3 gene. Examples of ³²P-labeled DNA probes which can be used in the Northern blot analysis are specifically detailed in Levanon et al., 1994, Genomics 23, 425-432). RNA from cells used in the assay is separated on an agarose gel and the separated RNA transblotted onto a nylon or other suitable membrane. The membrane-bound RNA is probed with specific nucleic acid sequences which will bind to the mRNA encoding the amino acid sequences of RUNX3. The probes are labelled, e.g., with. ³²P, to allow detection of probe binding to the appropriate mRNA. However, non-radioactive labeling and detection procedures may be used. An example of a non-radioactive labeled probe is one wherein the probe sequence includes digoxigenin (DIG)-labeled deoxyuridyltriphosphate (dUTP). The single stranded DIG-dUTP-labeled probes hybridize with the nucleic acids on the nylon membrane under conditions where the temperature and salt concentrations are carefully controlled. After several washing steps at different levels of stringency, the blot is developed using an anti-DIG antibody followed by color development steps.

In another embodiment, in situ hybridization may be used that allows the direct visualization of cellular mRNA levels in cultured cells or tissue sections (Remick, D. G., et al., Lab. Invest. 59:809 (1988)). The relative expression of mRNA in different samples can be determined. Cell samples are affixed to microscope slides using standard methods and reagents. The fixed cells are incubated in ethanol, and the sample is hybridized with a DNA probe specific for RUNX3 by placing a small volume of the probe on the slide, covering the slide, and incubating the slide overnight in a humidified atmosphere. The probes are labeled, typically with. ³⁵S, but non-radioactive probes labeled with DIG-dUTP as described can also been used. After hybridization, the slides are carefully washed under stringency conditions to remove all non-bound material, and the probe is visualized. For example, where the label is ³⁵S, the slides are covered with a photographic emulsion and developed after a week-long exposure. For DIG-labeled probes, a color development procedure is performed. After counterstaining with hematoxylin, the distribution of the probe can be visualized at the level of the light microscope.

In yet another embodiment, RT-PCR may be used which is a very sensitive and powerful technique for assessing mRNA levels is reverse transcriptase-polymerase chain reaction (RT-PCR, see, e.g., Erlich, PCR Technology (Freeman 1992), and Kilgus, O., et al., J. Invest Dermatol. 100:674 (1993), and U.S. Pat. Nos. 4,683,195, 4,683,202, 4,965,188). In this procedure, total RNA is isolated from a sample and the mRNA copied to DNA (cDNA) using reverse transcriptase. This cDNA is then added to a PCR reaction containing DNA primers which specifically target the mRNA species of interest. This PCR product is electrophoresed on an agarose gel, stained with a fluorescent dye, and photographed. The intensity of the staining of the PCR product is proportional to the concentration of the product and can be quantitated using a densitometer. By normalizing the expression of various genes based on β-actin expression, semi-quantitative determination of mRNA concentrations can be achieved by RT-PCR.

Polyclonal or monoclonal antibodies specific for RUNX3 expression product may be used in screening immunoassays. The term “antibody” is used in the broadest sense and specifically covers single monoclonal antibodies (including agonist and antagonist antibodies) and antibody compositions with polyepitopic specificity. The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, the monoclonal antibodies are advantageous in that they can be synthesized by hybridoma culture, uncontaminated by other immunoglobulins.

According to the invention, a sample is taken from a patient suspected of having a chronic inflammatory disease. Samples, as used herein, include biological fluids such as tracheal lavage, or bronchoalveolar lavage, blood, cerebrospinal fluid, tears, saliva, lymph, dialysis fluid and the like; organ or tissue culture derived fluids; and fluids extracted from physiological tissues. Also included in the term are derivatives and fractions of such fluids. Biopsy samples are of particular interest, e.g. trachea scrapings, etc. The number of cells in a sample will generally be at least about 10⁴ more usually at least about 10⁵. The cells may be dissociated, in the case of solid tissues, or tissue sections may be analyzed. Alternatively a lysate of the cells may be prepared.

Diagnosis may be performed by a number of methods. The different methods all determine the absence or presence of RUNX3 expression product in patient cells. For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescent markers, chemiluminescent markers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the RUNX3 expression product in a lysate. Measuring the concentration of RUNX3 expression product binding in a sample or fraction thereof may be accomplished by a variety of specific assays. A conventional sandwich type assay may be used. For example, a sandwich assay may first attach Runx3-specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of normal and/or abnormal Runx3 is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind Runx3 with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material. The signal produced by the bound conjugate is detected by conventional means. Where an enzyme conjugate is used, an appropriate enzyme substrate is provided so a detectable product is formed.

In another embodiment, Western blot analysis or other immunoassays using an antibody against the protein encoded by the RUNX3 gene may be employed as an alternative or additional method for determining the presence of the Runx3 protein product in the sample. This includes the direct measurement of protein expression using methods such as (1) ELISA; (2) radioimmunoassay (3) gel electrophoresis or western blotting, or (4) immunohistochemistry. Antibodies specific for Runx3 protein may be used in various immunoassays well known in the art. In one embodiment, polyclonal rabbit anti-Runx3 antibodies are used to determine the level of Runx3 protein (Levanon et al., Mech. Dev., 109, 413-417, 2001). For example, the antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Experimental Methods

### FACS Dot Plot Analysis by Gating the Most Mature HSA^{Low}TCR^{High} T Lymphocytes:

Splenocytes were stained for 30 min on ice with biotin conjugated anti CD4, Cy-chrome anti CD8, fluorescein isothiocyanate (FITC)-conjugated TCR and phycoerythrin (PE)-conjugated HSA, washed twice with 2 ml FACS buffer (0.5% BSA/0.1% NaN₃/PBS) by centrifuging 5 min 1200 rpm each time and then stained with streptavidin-APC 30 min on ice. Cells were washed again and fixed with 1% paraformaldehyde until analyzed. All antibodies were from Pharmingen. The analysis was carried out using a FACScan flow cytometer equipped with CellQuest software (Becton Dickinson).

Determination of CD4+/CD3+ Levels in Peripheral blood Illustrated:

Peripheral blood was collected from the heart of anesthesized mice and then subjected to a Ficoll-Paque (Amersham Pharmacia Biotech) gradient centrifugation (20 min at 1000 rpm) to isolate peripheral blood lymphocytes. Lymphocytes were washed twice in FACS buffer (0.5% BSA/0.1% NaN₃/PBS) by centrifuging 5 min 1200 rpm at 4° C. each time and then stained with Rat Anti-Mouse CD4-FITC (L3T4), CD3-SPRD and CD8-PE antibodies (all from Southern Biotech, Inc) for 30 min on ice. Cell were then washed twice with FACS buffer and fixed by gently vortexing each sample with 1% Paraformaldehyde/PBS. Cells were incubated overnight at 4° C. and analyzed the next morning using the FACScan flow cytometer equipped with the CellQuest Software (Becton Dickinson).

### Preparation of BALF (Bronchoalveolar Lavage Fluid) of RUNX3 KO Lungs:

Mice were sacrificed by cervical dislocation. The trachea was exposed and a cannula was inserted and secured by sutures. The lungs were lavaged four times by institution of 1 ml of ice-cold PBS and gently aspirating the fluid back. The recovered BALF was centrifuged (1000 rpm for 10 min at 4⁰cel), the cell pellet was used for differential counting and FACS analysis and the supernatant were frozen on dry ice and stored at −80⁰cel until use.

### Mice Strains and Treatments:

RUNX3-KO mice were generated as described previously (Levanon et al., 2002) and bred on ICR and MFI background. CX₃CR1^{+/GFP} mice (Jung et al., 2000) used to identify GFP⁺ DC populations were kept on C57BL/6 background. Mice were maintained in individually ventilated cages in an SPF facility free of known viral and bacterial pathogens. For bronchoalveolar lavage (BAL), 8-9 week old RUNX3 KO and age/sex matched WT littermate mice were sacrificed by CO₂ asphyxiation, tracheae were cannulated and lungs washed by gentle infusion of 24 aliquots of 1 ml PBS. Experimental acute asthma was induced in C57BL/6 mice by 3 weekly intraperitoneal injections of OVA in alum. Subsequently, mice were subjected to five daily 5% nebulized OVA inhalations. Four hours after the last inhalation, mice were sacrificed and BAL was performed. For the suboptimal OVA sensitization procedure, mice (RUNX3 KO and WT littermates) were subjected to a single OVA/alum injection, which 2 weeks later was followed by OVA inhalations. Peritonitis for neutrophil isolation was induced in WT mice by intraperitoneal injection of 3 ml 10% sodium caseinate. Sixteen hours later mice were sacrificed peritoneal lavage was performed and cells were morphologically identified and counted on cytospin preparations stained with May-Grunwald Giemsa. Analysis of LC was preformed using ear epidermal sheets prepared by splitting the ear and placing its dermal side down in 1% trypsin solution for 45 min at 37° C. Epidermal sheets were peeled off the underlying dermis and subjected to further analysis.

### Lung Histology:

Lungs were inflated with, and immersion fixed in 10% neutral buffered formalin. Tissue was processed routinely, embedded in paraffin, and trimmed at 4 μm. Selected cases were stained with periodic acid Schiff (PAS) and Masson's trichrome. The identity of eosinophils was confirmed using the phenol red staining procedure. Nuclei were counterstained with DAPI.

### Bone Marrow Cultures:

Bone marrow derived dendritic cells (BMDC) were prepared as described (Inaba et al, 1992). Briefly, mice were sacrificed and BM was extracted from femurs and tibias by flushing the shaft with 5 ml RPMI-1640. Red blood cells (RBC) were lysed in 1.66% NH₄Cl, and cells seeded into non-tissue culture plates at a density of 1×10⁶ cells/ml in medium (RPMI-1640, 5% FCS, 5×10⁻⁵ M 2-mercapto ethanol, penicillin/streptomycin) containing 10 ng/ml murine recombinant GM-CSF (Peprotech, Rehovot, Israel). Medium was replenished every three days and the loosely adherent DC were collected at designated time-points and used for further studies. To induce DC maturation, day 7-12 cultures were treated with LPS (0.1-1 μg/ml) and analyzed one day later.

### Gradient Enriched Splenic DC:

Spleens were isolated, minced and incubated with 1 mg/ml collagenase (Sigma) for 45 minutes at 37° C. RBC were lysed and cells resuspended at a density of 5×10⁷ cells/ml in a 14.5% Nycodenz solution (Nycomed, Pharma AS diagnostics, Oslo, Norway). Buffer (2 ml) was layered carefully onto 4 ml of Nycodenz cell suspension, centrifuged (1500 g×13 min at 4° C.) and the low-density cell layer was collected for further experiments.

### Flow Cytometry and ELISA:

Single cell suspensions were prepared in FACS buffer (PBS, 1 mM EDTA, 1% BSA/0.05% sodium azide) and filtered through an 80-μm nylon mesh. Cells were washed twice and counted with trypan blue for exclusion of dead cells. Immunostaining (1 to 2×10⁶ cells) was performed in the presence of rat anti-mouse Fc gamma RIII/II receptor (CD16/32; clone 2.4G2, ATCC), by incubating the cells with monoclonal antibodies for 30 min on ice (100L per 1×10⁶ cells). Cells were washed and resuspended in 0.3-0.5 ml for FACS analysis. Flow cytometry was performed with a FACSCalibur (Becton Dickinson, Mountainview, Calif.) equipped with a CellQuest software (Becton Dickinson) and cell sorting was performed with a FACS sorter (Vantage). Staining reagents included, CD8-Percp, CD 11c APC/PE, CD11b PE/FITC, CD11a/FITC, IA/IE PE, IAb FITC, CD80 FITC, CD86 FITC, strepavidin APC/PE, biotinylated OX40L, CD3, CD4 (Pharmingen, San Diego, Calif. USA). IL5 levels in BAL were determined using mouse IL5 ELISA detection kit (Duoset, Minneapolis Minn., USA).

### MLR and Syngeneic Oxidized T-Cell Proliferation Assays

Cells were obtained from spleen and lymph nodes of RUNX3 KO and WT littermate ICR mice as well as from three MHC haplotype-mismatched inbred WT strains (C57/BL/6 (H2^{b}), BALB/C(H2^{d}), and SJL (H2^{s}). CD4⁺ T cells were isolated by MACS columns (Miltenyi Biotec, Bergish Gladbach, Germany) using the manufacturers recommended conditions yielding ˜95% pure CD4⁺ T cells. Syngeneic T cells of KO and WT littermates were oxidized by 15 min incubation on ice in 0.25 mg/ml sodium periodate, as previously described. Purified CD4⁺ T cells, either syngeneic sodium periodate treated or cells from each of the three H2 haplotype mismatched WT strains, were incubated at 37° C. in flat bottom microtiter plates, at a final volume of 0.2 ml, with increasing numbers of splenic Nycodenz density gradient-enriched DC. After incubation for 1-3 days, 10 μl/well of thymidine (0.05 μCi/μl in oxidative mitogenesis or 0.1 μCi/μl in MLR) were added and incubation at 37° C. continued for 14 h and 8 h in oxidative mitogenesis and MLR, respectively. Cells were collected and incorporated radioactivity was determined.

### Immunohistochemistry

BMDC (CD11c) were sorted into mature (MHC II high) and immature (MHC II low) subset populations and Runx3 was detected as previously described using affinity purified rabbit anti RUNX3 (Levanon et al., 2001a). For cryostat sections, tissues were fixed with 4% paraformaldehyde to preserve GFP. Sections (12-16 μm) were stained overnight with mouse monoclonal anti GFP antibodies, (clone B34, Babco, Richmond, Calif.) and rabbit anti RUNX3 antibodies washed and reacted for 2 h at room temperature with fluorochrome-labeled secondary antibodies (488 goat anti mouse and 568 goat anti rabbit, Molecular Probes, Eugene, Oreg.). Data was acquired by fluorescent and confocal microscopy.

### Example 1

### RUNX3 KO Mice Develop a Perturbed Distribution of CD4/CD8⁺ T Lymphocytes

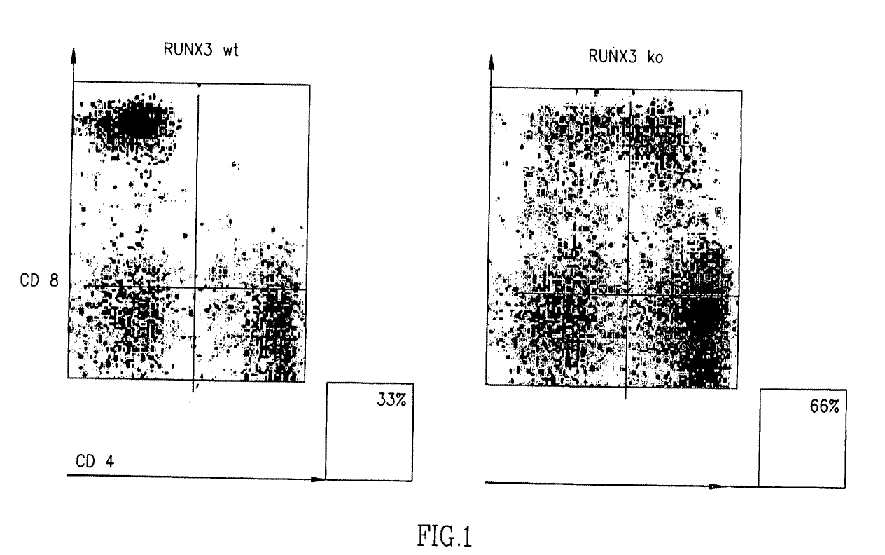

Mice lacking functional Runx3 protein (RUNX3 KO mice) were generated as described (Levanon et al. 2002). Phenotypically, RUNX3 KO mice exhibit heavy breathing and at accelerated rate, which was also associated with anxiety. FACS analysis revealed that these mice exhibit a perturbed distribution of CD4+/CD8+ T lymphocytes (TLs) in the thymus and spleen. An increase in the CD4+ subset and a decrease in the CD8+ TL subset are observed both in thymus and spleen. Specifically, a two-fold increase in the number of mature CD4+ cells was observed by gating in on the most mature HSA^{Low}TCR^{High} TLs in the spleen (FIG. 1). Similar results were obtained in peripheral blood T cells (Table 1).

TABLE 1
Percent of mature CD4⁺/CD3⁺ in peripheral
blood of ~6 week old WT and RUNX3-KO mice.

| | CD4^{+/}CD3⁺ |
|---|---|
| Genotype | (% of total T cells in blood) |
| WT | 18.22 |
| WT | 21.07 |
| WT | 10.77 |
| RUNX3-ko | 55.60 |
| RUNX3-ko | 63.54 |
| RUNX3-ko | 49.32 |

Double-sided p-value = 0.0299

### Example 2

### RUNX3 KO Mice have a Significantly Increased Level of IL-5

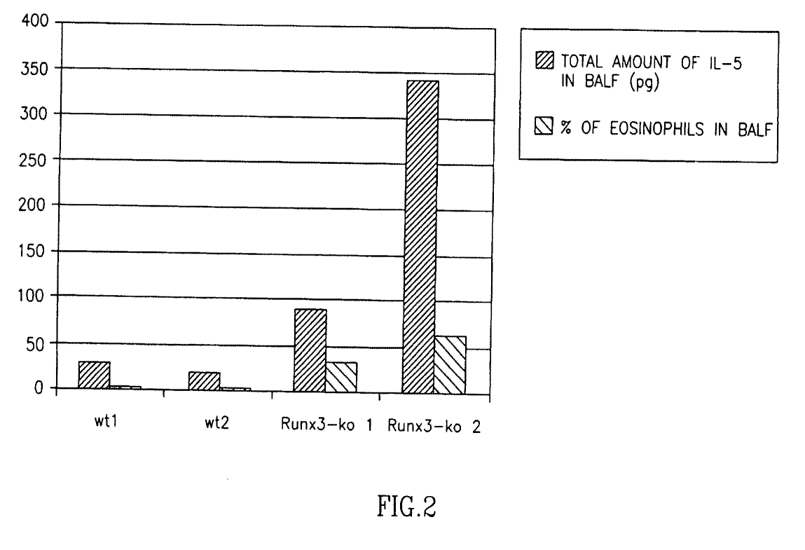

Bronchoalveolar lavage fluid (BALF) of RUNX3 KO and WT mice were analyzed by ELISA to determine the levels of IL-5. It was found that two RUNX3 KO mice (which had also 32% and 62% increase in eosinophils level in their BALF) had a significantly increased level of IL-5 (total amount of 90.4 pg and 342.5 pg, respectively), as compared to less than 30 pg IL-5 found in WT mice (FIG. 2).

Eosinophilic lung inflammation is commonly observed in atopic and non atopic asthma and IL-5 production by activated CD4+ T cells is enhanced in both atopic and non atopic patients as compared to normal control subjects implying that asthma may be a T cell disorder. Therefore, the above data indicate that RUNX3 KO mice develop a condition with characteristic features of asthma and may therefore serve as an animal model for the disease.

### Example 3

### RUNX3 KO Mice Develop Spontaneous Eosinophilic Airway Inflammation

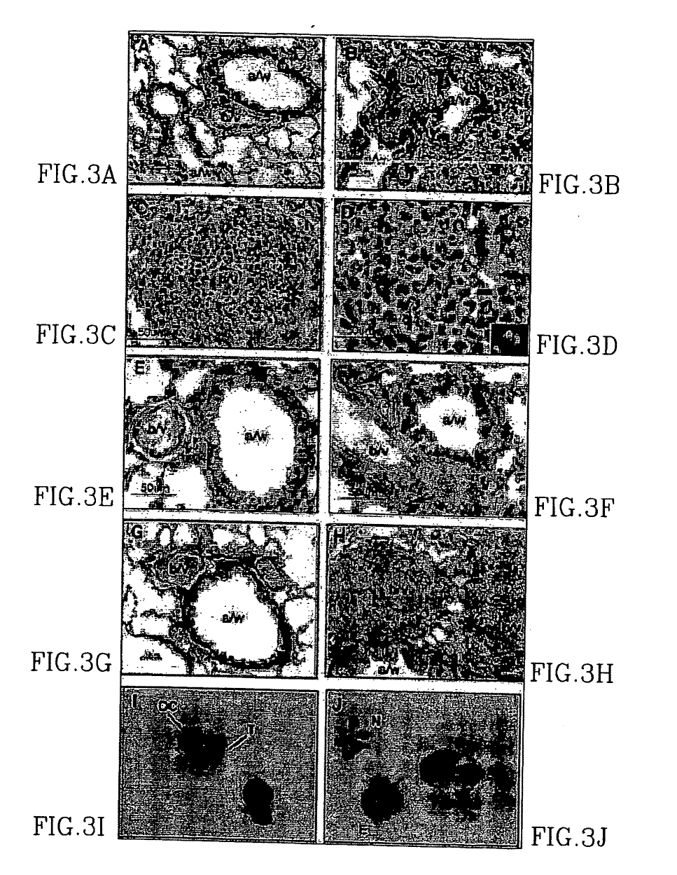

Infiltration of eosinophils was detected in lungs of 52% (20/38) of 1-8 week-old naïve KO mice, and in none of the WT controls. The eosinophilic infiltration was admixed with histiocytes and, less commonly, lymphocytes. Infiltrating cells were most consistently encountered in the interstitium around blood vessels and airways (FIGS. 3A and B), typically involving 2-3 lung lobes. Vascular cuffs composed of an essentially purely eosinophilic population were present in many KO mice (FIGS. 3C and D). In cases with more extensive cellular infiltration, eosinophils and mononuclear cells expanded into the adjacent alveolar septae and filled the alveolar spaces. In some cases, perivascular and peribronchial inflammatory infiltration was accompanied by hyperplasia of the airway mucosa, hypersecretion of mucus and excess deposition of collagen (FIG. 3E-H) indicating airways remodeling. Necropsies of older KO mice (2-16 month-old, n=12) revealed only 2 more cases of eosinophilic pneumonia indicating that the inflammatory infiltration is transient.

### Example 4

### Analysis of Bronchoalveolar Lavages (BAL) of WT and RUNX3 KO Mice

The eosinophilic lung inflammation in the KO mice led us to examine the cellular content of BAL from RUNX3 KO and WT mice. Total cell-count revealed a significant increase of cell number in KO compared to WT BAL (1.1±0.18×10⁶ vs. 0.2±0.09×10⁶ cells/BAL, respectively n=6, p=0.0016). Moreover, analysis of the BAL cell composition showed a marked preponderance of eosinophils in the KO compared to WT littermates (28.6±9.1% vs. 1.16±0.4%, respectively n=6, p=0.027). This alveolar eosinophilia in the KO was accompanied by increased levels of IL-5 in the BAL fluid compared to WT controls.

These results raised the question of the possible cause for the eosinophilic infiltration in the KO. To address this issue an experimental, ovalbumin (OVA)-induced acute asthma model was used (Topilski et al., 2002) to identify the RUNX3 expressing cells in the alveolar space of the treated animals. WT mice sensitized by OVA were subjected to OVA inhalation and BAL cells were isolated and analyzed for RUNX3 expression (FIGS. 3I and J). BAL of the OVA treated mice contained an abundance of conjugates between large mononuclear phagocytes and T cells that were not present in BAL of untreated mice. The latter were identified as CD4⁺ T cells by FACS analysis. Significantly, within each conjugate, RUNX3 expression was detected only in the mononuclear phagocyte; the T-cell was negative, as were BAL eosinophils and neutrophils (FIG. 3J). These results demonstrate that upon allergic sensitization alveolar DC or macrophages express high levels of Runx3. This could argue that Runx3 has an intrinsic function in these cells and might be involved in the etiology of OVA-induced pulmonary eosinophilia. Consistent with this thesis is the earlier finding that when Runx3 is lost the KO mice develop spontaneous pulmonary eosinophilic inflammation without any allergic sensitization.

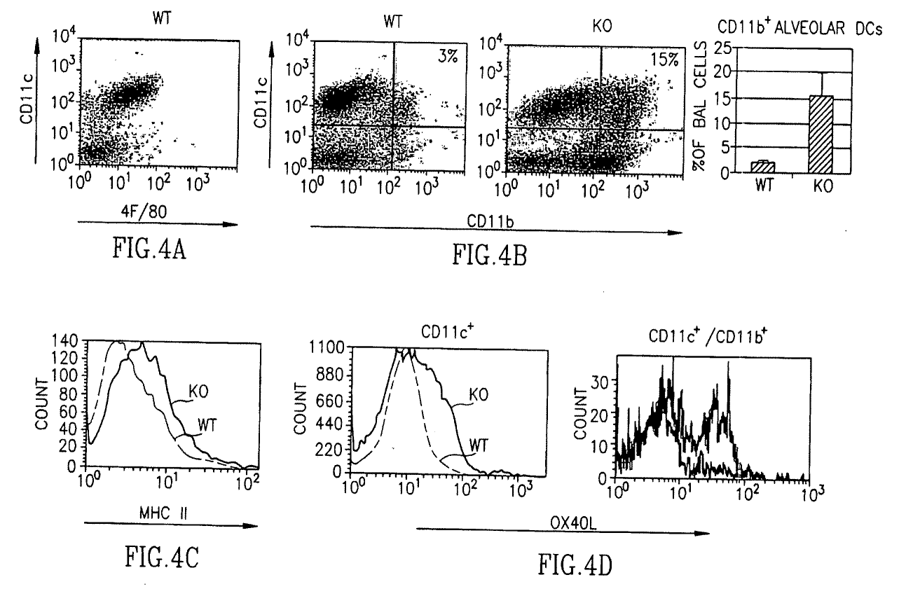

In steady state the murine lung alveolar space is populated by alveolar macrophages, which notably co-express the macrophage marker F4/80 and the DC marker CD11c (FIG. 4A) and are further characterized by high autofluorescence (Vermaelen et al., 2001). As such, these cells are distinct from monocytes and DC in the respiratory epithelium and lung parenchyma. Alveolar macrophages are poor T cell stimulators and are believed to be under constant immunosuppression by cytokines such as IL-10. More recently Julia et al (Julia et al., 2002) described an additional F4/80⁺/CD11c⁺/CD11b⁺ mononuclear phagocyte subset—referred to as alveolar DC—that represents a small population in the resting lung, but accumulates in inflammation. Alveolar DC are potent APC and have a sustained allergen presentation capacity (Julia et al., 2002). It remains unknown whether these alveolar DC arise from resident cells in the lung or are descendants of monocyte infiltrates.

To further evaluate the nature of the alveolar RUNX3-expressing mononuclear phagocytes, the DC/macrophages populations in BAL of untreated WT and KO mice were analyzed by flow cytometry. Analysis of KO BAL revealed a striking elevation of the F4/80⁺/CD11c/CD11b subset of alveolar DC as compared to WT BAL (FIG. 4B). Furthermore, CD11c⁺ BAL cells in the KO mice were characterized by increased expression of MHC II (FIG. 4C). Recently OX40L was shown to play a critical role in the development of allergic lung inflammation (Akbari et al., 2003). Compared to WT DC, a remarkable increase in expression of this costimulatory molecule was observed on RUNX3 KO BAL DC (FIG. 4D). Moreover, OX40L was also elevated on the CD11⁺/CD11b⁺ subset of alveolar KO DC (FIG. 4D).

To examine the response of RUNX3 KO and WT mice to aerosol inhalation, mice were next challenged with suboptimal doses of OVA. A marked increase in the F4/80⁺/CD11c/CD11b alveolar DC subset was observed in KO BAL compared to WT (0.58×10⁶±0.1 KO vs. 0.035×10⁶±0.01 WT, n=4, p=0.009) along with a two-fold increase in KO BAL eosinophils. Together, these data demonstrate that Runx3 deficiency results in accumulation of F4/80⁺/CD11c⁺/CD11b⁺/OX40L^{high} subset of alveolar DC. As highly potent APC, these cells may be responsible for the observed eosinophilic lung inflammation in the RUNX3 KO mice.

### Example 5

### Induction of RUNX3 Expression During DC Maturation

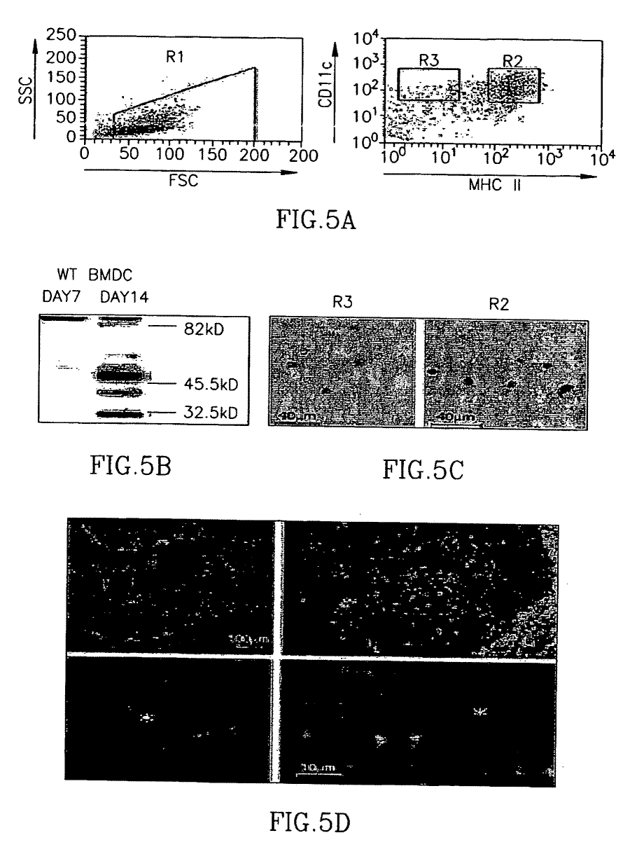

The expression of RUNX3 in the lung DC led us to examine its expression in other DC populations. To this end, mature DC from bone marrow of RUNX3 KO and WT littermates were generated (FIG. 5A). The non-adherent fraction of day 7th cultured WT bone marrow DC (BMDC) consisted of a mixed population of immature DC and granulocytes (not shown). At day 11 to day 14, the culture consisted of immature—and spontaneously matured—DC (˜66% and ˜33%, respectively). Expression of RUNX3 was not detected in day 7 immature DC, but was readily detected when the percentage of mature DC increased (FIG. 5B). More convincingly, when LPS treated BMDC cultures were sorted by FACS into mature and immature populations (R2 and R3 in FIG. 5A, respectively), collected onto glass slides and immunostained with anti Runx3 Ab, expression was detected only in mature and not in immature DC (FIG. 5C).

Of note, three RUNX3 protein bands were detected in mature WT DC (FIG. 5B). In addition to the two known bands of ˜48 and 46 kDa that correspond to the full-length RUNX3 proteins (Bangsow et al., 1998), a third Runx3 protein of ˜33 kDa was also detected.

To assess the expression of RUNX3 during in vivo maturation of DC, a mouse strain (CX₃CR1^{+/GFP}) was used, in which splenic DC are homogenously green fluorescent labeled (Jung et al., 2000). The vast majority of DC in mouse spleen are considered immature and located in the white pulp marginal zones. Hence, most green fluorescent DC in the CX₃CR1^{+/GFP} mice are found at the inter-phase between the red and white pulps. Upon LPS injection, these cells migrate from the marginal zone to the periarteriolar lymphoid sheath (PALS); the T-cell zone (Jung et al., 2000). Neither the propensity of the DC to migrate nor their function (IL-12 production) are impaired in CX₃CR1^{+/GFP} mice (Jung et al., 2000).

CX₃CR1^{+/GFP} mice were injected with LPS and 6 h later mice were sacrificed and cryo-sections of paraformaldehyde fixed spleens were stained for Runx3 (FIG. 5D). No Runx3 was detected in the marginal zone, consistent with the lack of RUNX3 expression in immature DC, whereas many GFP-positive and GFP-negative cells in the PALS expressed RUNX3 (FIG. 5D). These RUNX3 expressing PALS cells represent both GFP/T cells and GFP/mature DC. Taken together, the results of RUNX3 expression in sorted mature versus immature BMDC and splenic DC in situ studies demonstrate that Runx3 is upregulated in DC during the maturation process.

### Example 6

### RUNX3 KO DC Display Enhanced Maturation and Increased Potency to Stimulate T Cells

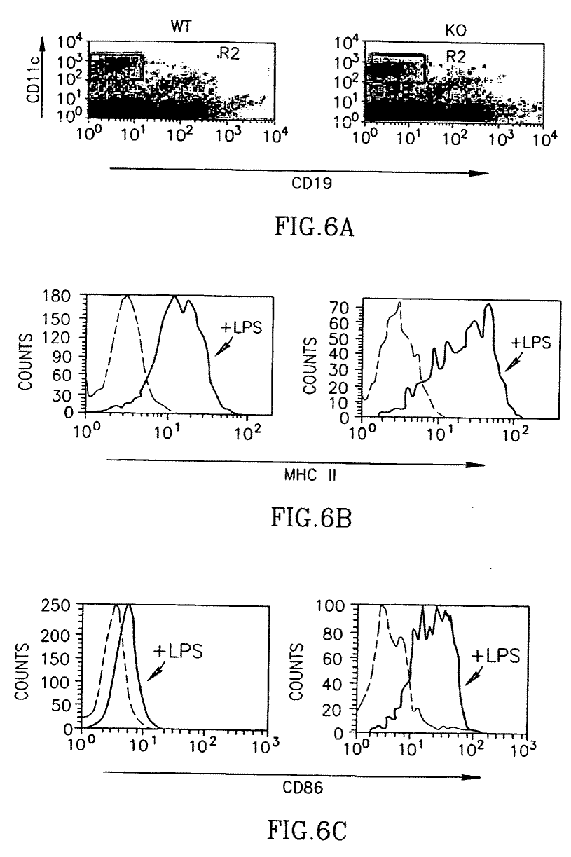

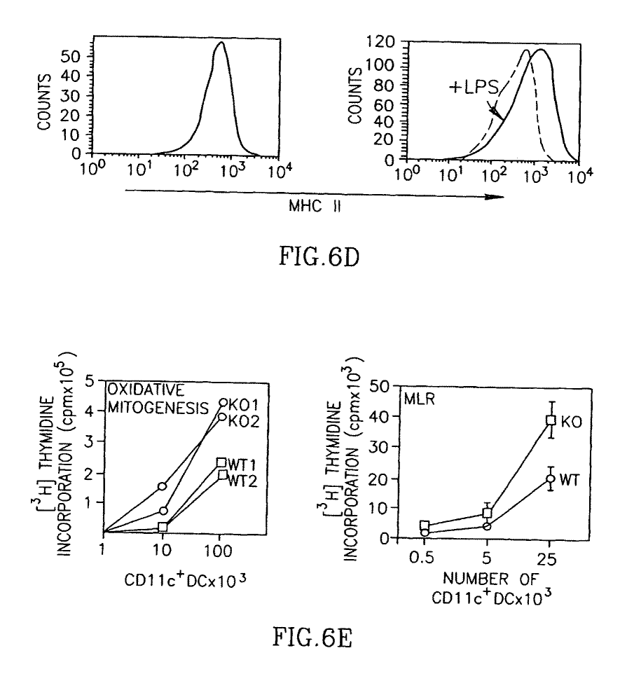

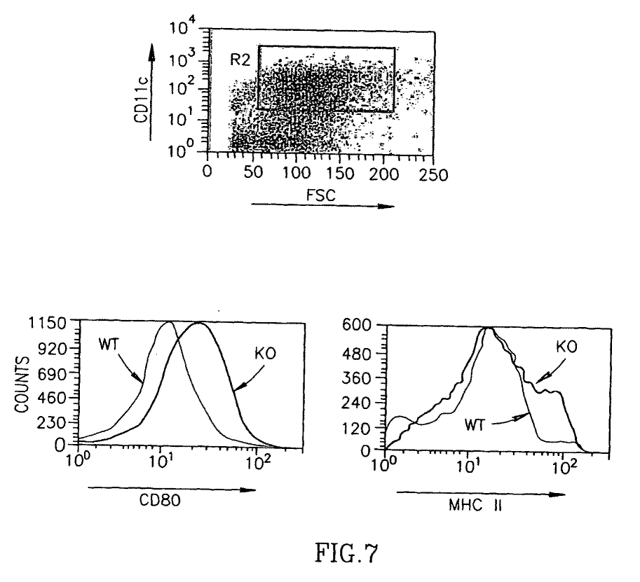

As shown above, the lack of Runx3 is associated with a large increase in mature DC in BAL of KO mice. The function of Runx3 in DC maturation was assessed. Density gradient enriched splenic DC were cultured overnight with maturation inducing reagents and analyzed by flow cytometry (FIG. 6A). High concentration of LPS (1 μg/ml) induced maturation of WT DC, reflected in elevated surface expression levels of MHC II and CD86 (FIGS. 6B, C). This LPS induced maturation was significantly more pronounced in the KO DC (FIGS. 6B, C). Of note, KO DC matured even at a suboptimal concentration of LPS (100 ng/ml), which did not affect WT DC (FIG. 6D). Experiments using other DC maturation-inducing-reagents, including TNFα and anti CD40 antibodies gave similar results (data not shown). Enhanced maturation was also found in RUNX3 KO BMDC as compared to WT cells (FIG. 7). The enhanced spontaneous maturation of the KO DC is evidenced by a larger proportion of cells with high CD80 and MHC II.

Contrary to immature DC that are not fully potent to prime T cells, mature DC are powerful stimulators of T-cell proliferation. It was further examined whether enhanced maturation of RUNX3 KO DC results in increased efficacy to stimulate T cells. To this end, the ability of WT and KO DC to stimulate T cells was examined using the syngeneic oxidative mitogenesis assay (Austyn et al., 1983) and a mixed leukocyte reaction (MLR). In both assays RUNX3 KO DC were significantly more efficient stimulators of CD4⁺ T-cell proliferation compared to WT DC (FIG. 6E). Taken together these data indicate a critical role for Runx3 in DC differentiation and function.

### Example 7

### Impaired TGF-β Signaling in RUNX3 KO DC

In vitro studies have previously shown that Runx3 participates in TGF-β directed immunoglobulin class switching to IgA by mouse splenic B cells (Shi and Stavnezer, 1998). More recently, Runx3 was identified as a mediator of both growth inhibition and apoptosis inducing activities of TGF-β in stomach epithelium (Ito et al., 2003; Li et al., 2002). In the DC compartment, TGF-β is known to play a dual role. First, TGF-β is absolutely essential for the development of the epidermal subset of DC, the LC (Borkowski et al., 1996), and second, TGF-β acts as a maturation inhibitor of DC (Yamaguchi et al., 1997).

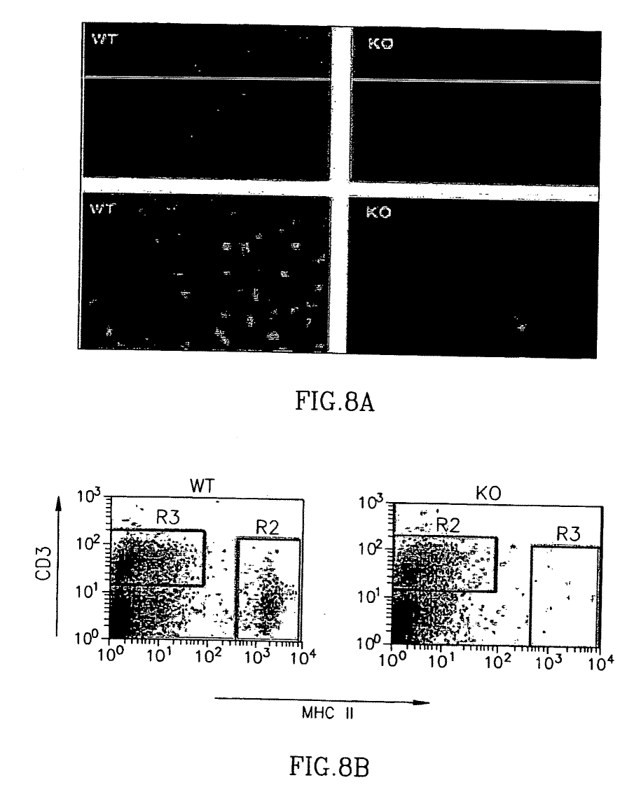

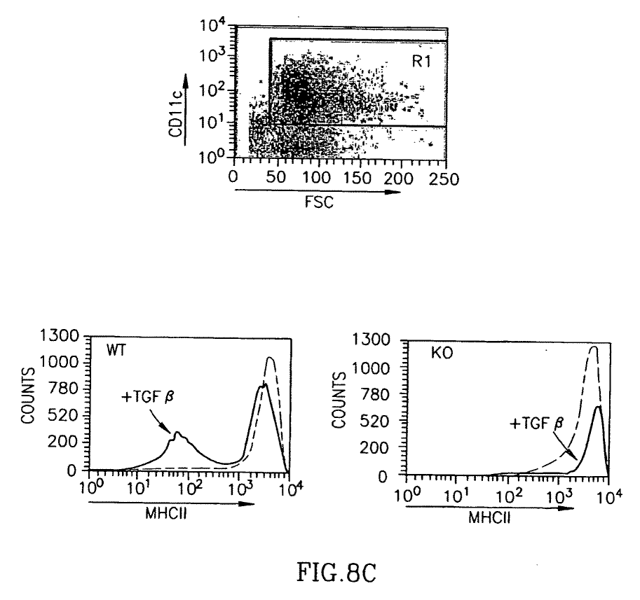

To investigate the effect of Runx3 deficiency on TGF-β signaling in DC, LC development in RUNX3 KO mice was assessed. When epidermis of WT and KO mice was analyzed it was found that RUNX3 KO mice completely lack epidermal LC (FIG. 8A), whereas the abundance of other epidermis constituents, such as CD3⁺ T cells, was similar (FIG. 8B). Next, the effect of TGF-β on maturation of RUNX3 KO and WT DC was examined. As shown in FIG. 8C, TGF-β inhibited the LPS-induced maturation of WT BMDC, but strikingly failed to do so in KO BMDC. The results indicate that Runx3 functions as part of the TGF-β signaling pathway in DC.

In vitro studies have previously shown that Runx3 participates in TGF-β mediated immunoglobulin class switching to IgA by mouse splenic B cells (Shi and Stavnezer, 1998). We therefore examined the TGF-β mediated class switching to IgA in WT and RUNX3 KO splenic B cells and also measured the in vivo production of IgA. Splenocytes from KO and WT mice were incubated in the presence of TGF-β and LPS and after 4 days analyzed for the presence of germline (GL) Ig α and rearranged IgA transcripts by RT-PCR using the primers: IgA germline (IgA GL) Forward 5′-CCTGGCTGTTCCCCTATGAA-3′ (denoted as SEQ ID No. 1) Reverse 5′-GAGCTGGTGGGAGTGTCAGTG-3′ (denoted as SEQ ID No. 2);

IgA post switch (ps IgA) Forward 5′-CTCTGGCCCTGCTTATTGTTG-3′ (denoted as SEQ ID No. 3); Reverse 5′GAGCTGGTGGGAGTGTCAGTG-3′ (denoted as SEQ ID No. 4);
IgM Forward 5′-CTCTGGCCCTGCTTATTGTTG-3′ (denoted as SEQ ID No. 5);
Reverse 5′-GAAGACATTTGGGAAGGACTGACT-3′ (denoted as SEQ ID No. 6);
Actin Forward 5′-GATGACGATATCGCTGCGCTG-3′ (denoted as SEQ ID No. 7);
Reverse 5′-GTACGACCAGACGGCATACAGG-3′ (denoted as SEQ ID No. 8).

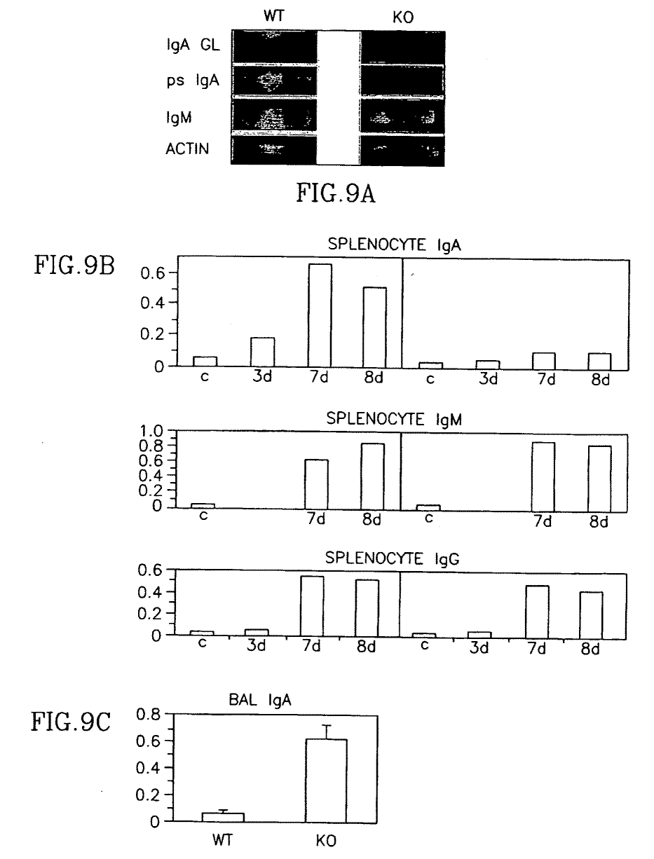

Significantly, GL and post-switch IgA (ps IgA) transcripts were observed in RNA isolated from WT splenocytes, but not in RNA from RUNX3 KO splenocytes (FIG. 9A), whereas IgM mRNA was readily detected in both. Consistent with these results, supernatants of cultured WT splenocytes contained higher levels of IgA than supernatants of the KO, whereas levels of IgM and IgG were similar (FIG. 9B). These results further support the findings that indicate a role for Runx3 as mediator of TGF-β signaling. Intriguingly, however, the level of IgA in BAL of RUNX3 KO mice was elevated (6 fold) as compared to WT (FIG. 9C) as were the levels of IgA in the serum and fecal pellets of the KO mice (data not shown). It thus appears that Runx3 is required for class switching to IgA in cultured splenocytes in vitro, but not for the production of IgA in vivo, indicating that more than one pathway may play a role in the switch to IgA.

### Example 8

### Altered Expression Pattern of 82-Integrins in RUNX3 KO DC

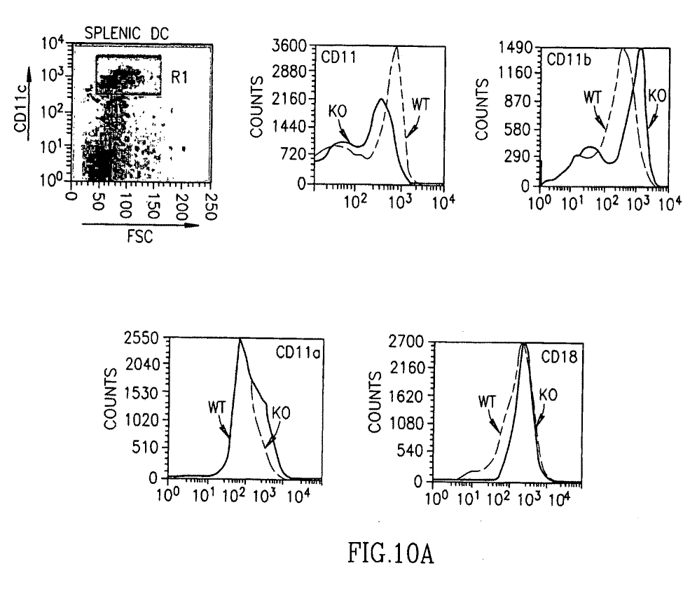

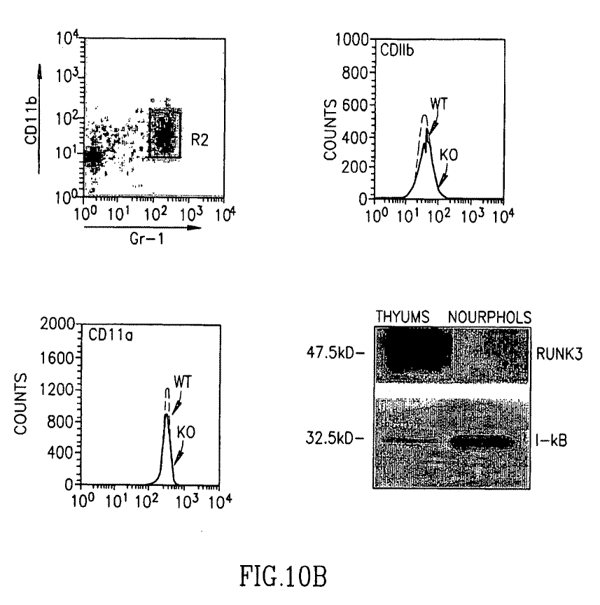

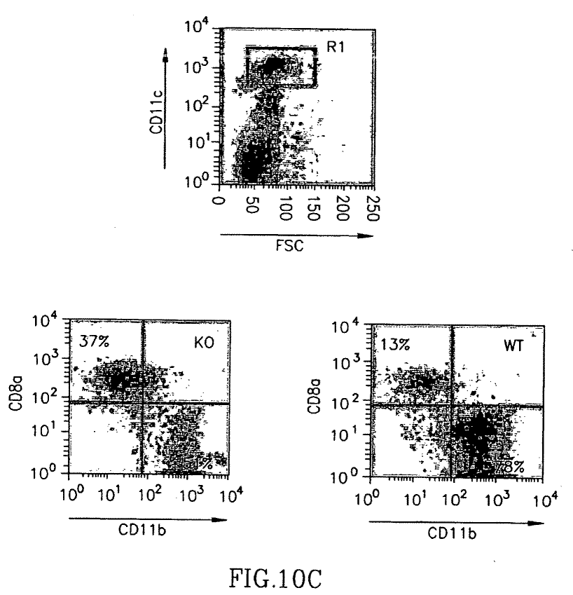

RUNX3 was recently implicated in regulation of lymphoid and myeloid specific activity of the CD11a promoter (Puig-Kroger et al., 2003). The enhanced maturation of RUNX3 KO DC described above, led us to test whether Runx3 deficiency effects expression of the β2-integrins in DC. Splenic DC of RUNX3 KO and WT littermates were analyzed by FACS for P2-integrins expression (FIG. 10A). The KO DC displayed marked reduction in CD11c, elevation of CD11b and a small increase of CD11a expression, as compared to WT DC (FIG. 10A). Expression of CD11c was also significantly reduced in DC of RUNX3 KO mesenteric lymph nodes and gut Peyer's patches as well as in RUNX3 KO BMDC (data not shown). Of note, expression levels of the 0 chain (CD18), which is common to all three β2-integrins, was not affected by the loss of Runx3 and was similar in KO and WT DC (FIG. 10A). These data indicate that Runx3 is directly involved in regulating the coordinated expression of β2-integrins. Consistent with this conclusion, the expression of β2-integrins in neutrophils, which do not express Runx3, was similar in both WT and KO (FIG. 10B).

Splenic DC fall into two distinct populations, defined by differential expression of CD11b and CD8α. Analysis of splenocytes from KO and WT littermates revealed a significant preponderance of the CD8⁺/CD11b DC in the KO compared to WT (35.0±4.1% vs. 15.5±2.1%, n=4; p=0.02), along with a decrease in the CD8CD11b⁺ DC (55.3±4.9% vs. 68.7±0.9%, n=4; p=0.04) (FIG. 10C). This altered population balance in splenic DC adds to the alterations in the KO DC compartment and may contribute to the increased inflammatory response in RUNX3 KO mice.

### Example 9

### RUNX3 KO Mice Develop Symptoms Characteristic of Idiopathic Inflammatory Bowel Diseases

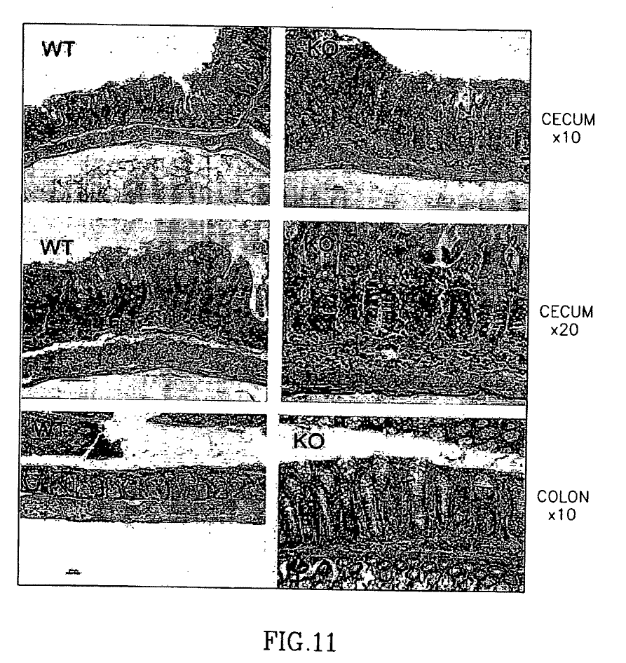

Beginning from approximately 5 month of age RUNX3 KO mice exhibit inflammatory cellular infiltration in the cecum, colon and rectum (typhlocolitis and proctitis, respectively (FIG. 11). The infiltrate is moderate to marked in extent, composed of plasma cells, lymphocytes, eosinophils and histiocytes and is limited to the mucosa-submucosa. Associated mucosal alterations include crypt loss, pronounced crypt elongation, and reduction in the number of goblet cells. The inflammatory process is segmental to diffuse. Peyers' patches and the mesenteric lymph nodes are reactive—they are enlarged and contain many secondary follicles with germinal centers. An accompanying mild lymphoplasmacytic enteritis, which is more severe than the ‘background’ mononuclear inflammatory infiltrate into WT small intestines, is observed in a minority of RUNX3 KO mice.

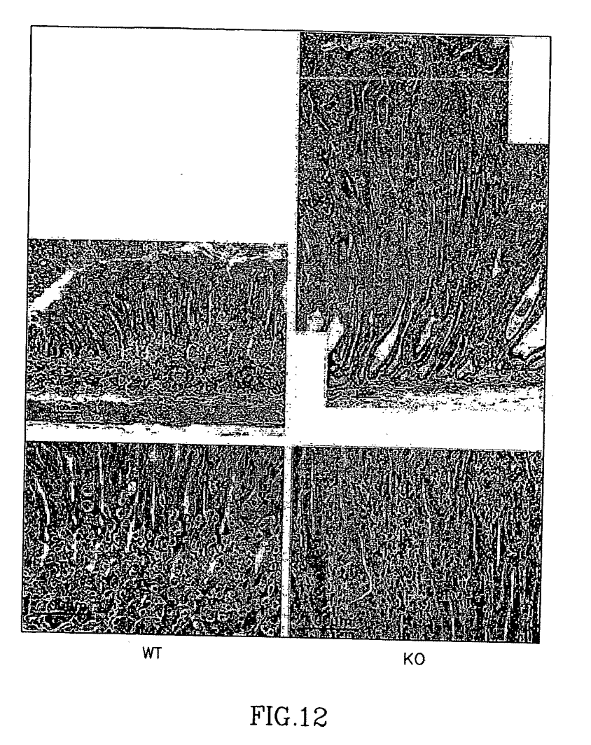

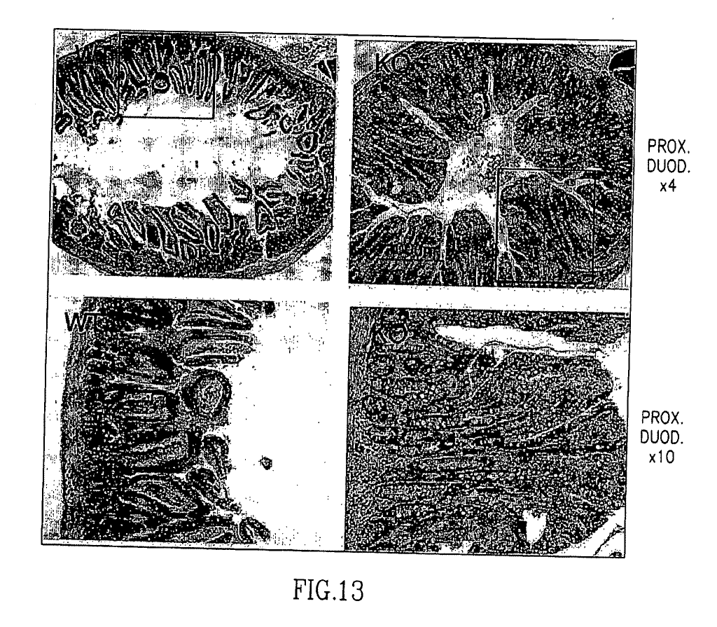

Concomitant with the above, there is pronounced hyperplasia of the glandular mucosa of the stomach of RUNX3 KO mice (FIG. 12). Hyperplastic pyloric/antral & fundic/oxyntic mucosa is taller than WT (to 3-fold) has a reduced complement of parietal and zymogen cells. Severely hyperplastic fundic mucosa is essentially devoid of parietal and chief cells and is comprised of tightly packed cuboidal and columnar cells some of which contain mucus. Additional mucosal changes include proliferation of columnar epithelial cells with expanded eosinophilic cytoplasm (hyalinosis), cyst formation with accumulation of crystalline secretory material, and in advanced cases, formation of adenomatous polyps in the pyloric region. The severity of hyperplastic changes correlates with the mouse's age, with older RUNX3 KO mice exhibiting more advanced hyperplasia. The hyperplastic changes begin in the antral mucosa and progress proximally to ultimately involve the entire fundic mucosa. Gastritis is modest and seen as multifocal mild to moderate mononuclear and eosinophilic infiltration of the mucosa and submucosa. An inflammatory infiltrate of similar composition is present in the proximal duodenum where it is associated with severe avillous hyperplasia (FIG. 13).

The three lesions (typhlocolitis, gastric mucosal hyperplasia/proliferative gastritis and proximal duodenitis) exhibit a temporal sequence of development. This is compatible with the typhlocolitis being the primary lesion, the gastric hyperplasia/proliferative gastritis secondary to the colitis (Reference: Fox, Dangler, and Schauer: Inflammatory bowel disease in mouse models: role of the gastrointestinal microbiota as proinflammatory modulators. In: Pathology of Genetically Engineered Mice, Ward et al. Editors. Iowa State University Press, Ames 2000), and the proximal duodenitis being a sequelum of the gastric alterations.

### Example: 10

### Summary of Analysis of Genetic Association of RUNX3 with Asthma in Humans

Five SNPs with no or limited linkage disequilibrium between them were genotyped on 600 asthma cases and 600 controls. These included two SNPs in the 5′ region of RUNX3, two in introns and one in the 3′ region.

Additional genotyping of two out of the five SNPs (one from the 5′ region of RUNX3 and one from the 3′ region) was carried out in Phase H of this study in order to bring the total number of cases to 1000 and controls to 1500.

There were no significant differences in allele frequencies or genotype distributions between cases and controls for any of the five SNPs examined. When the frequency of each of the three possible genotypes was compared between cases and controls for SNP 1-00029, in the 3′ region of RUNX3, the frequency of one homozygotic genotype was found to differ between all cases (558) and controls (591) with a p value=3.65×10⁻² and the heterozygotic genotype differed between male cases (266) and controls (591) with a p value=2.42×10⁻². These values may be viewed as marginally significant.

We analyzed the results of the additional cases and controls that were genotyped separately in order to establish whether our initial findings on SNP I-00029 would be independently repeated. Indeed, in our Phase II findings on 427 additional cases and 922 additional controls, the frequency of the same homozygotic genotype was again found to differ between all cases and controls, this time with a p value=4.27×10⁻². Unlike in Phase I, there was also a significant difference in allele frequencies of this SNP between cases and controls (p value=1.49×10⁻²) when the Phase II individuals were examined separately. Thus, SNP 1-00029 was found to be associated with disease in two independent comparisons between cases and controls.

When our Phase II results were combined with those of Phase I, the significance of findings for SNP 1-00029, in the 3′ region of RUNX3, was further increased. Firstly, the difference in the allele frequency of this SNP between 1030 cases and 1513 controls is significant at a p value=9.36×10⁻³ and between 590 female cases and 1513 controls at a p value=5.93×10⁻³. Secondly, when the frequency of each of the three possible genotypes was compared, the frequency of one homozygotic genotype differed between all the cases and the controls with a p value=6.10×10⁻³ (compared to a significance level of 3.65×10⁻² previously observed). Even considering the large number of tests that were conducted on the data (in all 75), these values suggest that SNP 1-00029 in RUNX3 may be associated with asthma.

When multiple SNPs were considered simultaneously, the haplotype giving the most significant result (p=1.73×10⁻², for all cases vs. control) was a two SNP haplotype comprised of SNP 1-00029, in the 3′ region of the gene, and SNP 1-00023, in the 5′ region.

Subsequent to the increase in the number of genotypes available, the significance of the two SNP haplotype comprised of SNP 1-00029, in the 3′ region of the gene, and SNP 1-00023, in the 5′ region was enlarged to a p value=2.48×10⁻³ for all cases (1022) vs. controls (1480), thus further supporting the notion of the disease association of the RUNX3 gene.

The association of each of the 5 SNPs to subcategories of patients was analyzed. Subcategories that include more than 200 individuals were considered since they are less prone to random differences. The overall indication from this analysis is that particular forms of 4 SNPs in RUNX3 are all consistently, but marginally, associated with a late onset, mild form of asthma in a gender independent manner. Thus, in all patients, SNPs 1-00023, 1-00026 and 1-00030 are associated with the occurrence of two or fewer episodes of asthma per week, SNP 1-00029 is associated with an age at diagnosis of greater than 12 years and both SNPs 1-00029 and 1-00030 are associated with the therapeutic use of corticosteroids at low to medium doses. While most p values are in the order of 10⁻², some do achieve significance levels of 10⁻³. As above, the issue of multiple testing must be considered when evaluating these significance levels.

With the additional data SNPs 1-00023 and 1-00029 display greater association than previously to the subcategory of patients suffering from a mild form of asthma. Thus, the allele frequency of SNP 1-00023 displays association with therapeutic use of low to medium doses of inhaled corticosteroids among patients (p value=9.74×10⁻³) and with the occurrence of 2 or fewer asthmatic episodes per week (p value=6.57×10⁻³) when patient subclasses are compared to controls. In the case of SNP 1-00029, both allele and genotype frequency differences for the same patient subcategories are in the range of p value=1 to 4×10⁻². This is also the only SNP which displays a similar low level of association with late age of diagnosis. However, the most significant scores for association of a single SNP to patient subclasses continues to be those of SNP 1-00030 which is, like SNP 1-00023, in the 5′ region of the gene. This SNP displays association with inhalation of low to medium doses of corticosteroids with an allele p value=4.30×10⁻³ and a score for one of the genotypes of p value=2.41×10⁻³.

Results of Additional Analyses

Additional statistical analyses were carried out to further characterize the association of RUNX3 with a late onset, mild form of disease.

First, the relationships between the phenotypic measurements that characterize different patient subcategories (age of disease onset, frequency of symptoms and dosage of inhaled corticosteroids) were examined. A strong correlation was found only for the relationship between frequency of symptoms and corticosteroid dosage (p value=9.52×10⁻⁴), suggesting that low symptom frequency and low corticosteroid dosage describes largely overlapping populations while patients with late onset of disease are a more distinct group for which the overall statistical evidence of association with SNPs in RUNX3 is smaller.

Next, patient subclasses were compared among themselves by haplotypic analysis. Haplotypic analysis was carried out with all combinations of SNPs 1-00023, 1-00026, 1-00029 and 1-00030 to determine if a) patients with early onset of disease bear haplotypes that are statistically different from patients with late onset, b) patients that take low to medium doses of corticosteroids are statistically different from patients that take high doses and c) patients who suffer from two or fewer asthmatic attacks a week are different from those who suffer from more than two attacks a week. Only the subclasses of patients who differ in their use of inhaled corticosteroids were found to be significantly different from one another when comparing all the haplotypic distributions in three 2-SNP haplotypes and one 3-SNP haplotype (lowest p value ˜9.16×10⁻³), all of which included SNP 1-00030. This additional analysis supports the notion that there is a strong association between SNP 1-00030 and therapeutic doses of corticosteroids.

Lastly, haplotype analysis with all possible combinations of SNPs 1-00023, 1-00026, 1-00029 and 1-00030 was carried out to compare each of the patient subclasses versus controls and provide further evidence for association of polymorphisms in the RUNX3 gene with a particular subclass of patient. Two 2-SNP haplotypes, one with SNPs 1-00023 and 1-00029 and one with SNPs 1-00029 and I-00030, gave globally significant results and were analyzed further. It was then demonstrated that the CA haplotype in SNPs 1-00023 and 1-00029 displayed significant association with late age of onset (p value=8.01×10⁻³), low or medium drug dosage (p value=4.28×10⁻³), and low symptom frequency (p value=3.9×10⁻⁴). The 2-SNP haplotype involving SNPs 1-00029 and 1-00030 gave less significant results (probably because of the smaller number of genotypes carried out on SNP 1-00030). In this case the AA haplotype displayed significant association with late age of onset (p value=2.66×10⁻²), low or medium drug dosage (p value=4.32×10⁻³), and low symptom frequency (p value=7.06×10³).

Summary and Discussion

In summary, there are some indications for the association of variations in the RUNX3 gene, particularly in regulatory regions of the gene, with a late onset, mild form of asthma. These observations may contribute to the notion of RUNX3 involvement in the disease when taken together with the biological understanding of RUNX3 function.

The additional data on SNPs 1-00023 and 1-00029 that has been generated in Phase II of this study lends further support to the association of variations in the RUNX3 gene and asthma. This conclusion is based on the independent evidence for association that was observed for SNP 1-00029 in Phases I and II and on the statistical significance of the 2 SNP haplotype including SNPs 1-00023 and 1-00029, both of which were genotyped in 1000 cases and 1500 controls, which yields a p value=2.48×10⁻³.

The association of a single polymorphism in RUNX3 with a particular patient subcategory, users of low to medium doses of inhaled corticosteroids, is strongest for SNP1-00030 (allele p value=4.30×10⁻³ and a score for one of the genotypes of p value=2.41×10⁻³). The association of additional SNPs to the same patient subclass may reflect their levels of Linkage Disequilibrium with SNP 1-00030. Thus SNP 1-00023 which is in highest LD with SNP 1-00030 is also, of all the remaining SNPs, most significantly associated with low to medium corticosteroid doses. This SNP also displays almost as high a level of association (allelic p value=6.57×10⁻³) with the related phenotypic trait, low symptom frequency.

The association of RUNX3 with the subclass of patients suffering from a mild form of the disease was further substantiated by haplotypic analysis. Probably because of the larger number of genotypes carried out on SNPs 1-00023 and 1-00029, a 2-SNP haplotype involving these SNPs yielded the highest association with patient subclasses reaching a level of p value=8.01×10⁻³ for late age of onset, p value=4.28×10⁻³ for low to medium drug dosage, and p value=3.9×10⁻⁴ for low symptom frequency. Lower, but significant values, were obtained for a 2-SNP haplotype involving SNPs 1-00029 and 1-00030, the latter of which was genotyped on fewer individuals but, on its own, demonstrated the highest association for a single SNP with drug dosage.

Taken all together, these findings support the notion of association of SNPs 1-00023, 1-00029 and 1-00030, all in the regulatory regions of the RUNX3 gene, to asthma, and, particularly to a mild form of asthma characterized by fewer attacks and lower drug dosage.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

### REFERENCES

Akbari, O., Stock, P., DeKruyff, R. H. and Umetsu, D. T. (2003) Mucosal tolerance and immunity: regulating the development of allergic disease and asthma. Int Arch Allergy Immunol, 130, 108-118.
Austyn, J. M., Steinman, R. M., Weinstein, D. E., Granelli-Pipemo, A. and Palladino, M. A. (1983) Dendritic cells initiate a two-stage mechanism for T lymphocyte proliferation. J Exp Med, 157, 1101-1115.
Banchereau, J. and Steinman, R. M. (1998) Dendritic cells and the control of immunity. Nature, 392, 245-252.
Bangsow, C., Rubins, N., Glusman, G., Bernstein, Y., Negreanu, V., Goldenberg, D., Lotem, J., Ben-Asher, E., Lancet, D., Levanon, D. and Groner, Y. (2001) The RUNX3 gene—sequence, structure and regulated expression. Gene, 279, 221-232.
Borkowski, T. A., Letterio, J. J., Farr, A. G. and Udey, M. C. (1996) A role for endogenous transforming growth factor beta 1 in Langerhans cell biology: the skin of transforming growth factor beta 1 null mice is devoid of epidermal Langerhans cells. J Exp Med, 184, 2417-2422.
Holt, P. G. (2000) Antigen presentation in the lung. Am J Respir Crit. Care Med, 162, S151-156.
Inaba, K., Inaba, M., Romani, N., Aya, H., Deguchi, M., Ikehara, S., Muramatsu, S. and Steinman, R. M. (1992) Generation of large numbers of dendritic cells from mouse bone marrow cultures supplemented with granulocyte/macrophage colony-stimulating factor. J Exp Med, 176, 1693-1702.
Ito, Y. and Miyazono, K. (2003) RUNX transcription factors as key targets of TGF-beta superfamily signaling. Curr Opin Genet Dev, 13, 4347.
Julia, V., Hessel, E. M., Malherbe, L., Glaichenhaus, N., O'Garra, A. and Coffman, R. L. (2002) A restricted subset of dendritic cells captures airborne antigens and remains able to activate specific T cells long after antigen exposure. Immunity, 16, 271-283.
Jung, S., Aliberti, J., Graemmel, P., Sunshine, M. J., Kreutzberg, G. W., Sher, A. and Littman, D. R. (2000) Analysis of fractalkine receptor CX(3)CR1 function by targeted deletion and green fluorescent protein reporter gene insertion. Mol Cell Biol, 20, 4106-4114.
Levanon, D., Bettoun, D., Harris-Cerruti, C., Woolf, E., Negreanu, V., Eilam, R., Bernstein, Y., Goldenberg, D., Xiao, C., Fliegauf, M., Kremer, E., Otto, F., Brenner, O., Lev-Tov, A. and Groner, Y. (2002) The Runx3 transcription factor regulates development and survival of TrkC dorsal root ganglia neurons. Embo J. 21, 3454-3463.
Levanon, D., Brenner, O., Negreanu, V., Bettoun, D., Woolf, E., Eilam, R., Lotem, J., Gat, U., Otto, F., Speck, N. and Groner, Y. (2001a) Spatial and temporal expression pattern of Runx3 (Aml2) and Runx1 (Aml1) indicates non-redundant functions during mouse embryogenesis. Mech Dev, 109, 413-417.
Levanon, D., Goldstein, R. E., Bernstein, Y., Tang, H., Goldenberg, D., Stifani, S., Paroush, Z. and Groner, Y. (1998) Transcriptional repression by AML1 and LEF-1 is mediated by the TLE/Groucho corepressors. Proc. Natl. Acad. Sci. USA, 2995, 11590-11595.
Levanon, D., Brenner, O., Otto, F. and Groner, Y. (2003a) Runx3 knockouts and stomach cancer. EMBO Rep, 4, 560-564.
Li, Q. L., Ito, K., Sakakura, C., Fukamachi, H., Inoue, K., Chi, X. Z., Lee, K. Y., Nomura, S., Lee, C. W., Han, S. B., Kim, H. M., Kim, W. J., Yamamoto, H., Yamashita, N., Yano, T., Ikeda, T., Itohara, S., Inazawa, J., Abe, T., Hagiwara, A., Yamagishi, H., Ooe, A., Kaneda, A., Sugimura, T., Ushijima, T., Bae, S. C. and Ito, Y. (2002) Causal relationship between the loss of RUNX3 expression and gastric cancer. Cell, 109, 113-124.
Nathan, C. (2002) Points of control in inflammation. Nature, 420, 846-852.
Puig-Kroger, A., Sanchez-Elsner, T., Ruiz, N., Andreu, E. J., Prosper, F., Jensen, U. B., Gil, J., Erickson, P., Drabkin, H., Groner, Y. and Corbi, A. L. (2003) RUNXJAML and C/EBP factors regulate CDI11a integrin expression in myeloid cells through overlapping regulatory elements. Blood.
Shi, M. J. and Stavnezer, J. (1998) CBF alpha3 (AML2) is induced by TGF-beta1 to bind and activate the mouse germline Ig alpha promoter. J Immunol, 161, 6751-6760.
Topilski, I., Harmelin, A., Flavell, R. A., Levo, Y. and Shachar, I. (2002) Preferential Th1 immune response in invariant chain-deficient mice. J Immunol, 168, 1610-1617.
Vermaelen, K. Y., Carro-Muino, I., Lambrecht, B. N. and Pauwels, R. A. (2001) Specific migratory dendritic cells rapidly transport antigen from the airways to the thoracic lymph nodes. J Exp Med, 193, 5160.
Yamaguchi, Y., Tsumura, H., Miwa, M. and Inaba, K. (1997) Contrasting effects of TGF-beta 1 and TNF-alpha on the development of dendritic cells from progenitors in mouse bone marrow. Stem Cells, 15, 144-153.

What is claimed is:

1. A method of testing efficacy of a treatment for a chronic inflammatory disease which comprises subjecting a mouse that is homozygous for a RUNX3 null allele to a putative treatment and determining the efficacy of such treatment by measuring severity of symptoms characteristic of the disease exhibited by the mouse, in comparison to severity of symptoms exhibited by the same mice not exposed to the treatment.

2. The method of claim 1, wherein the chronic inflammatory disease is an eosinophilic lung inflammation-related disease, a chronic obstructive pulmonary disease or an inflammatory bowel disease.

3. The method of claim 2, wherein the chronic obstructive pulmonary disease is chronic bronchitis.

4. The method of claim 2, wherein the eosinophilic lung inflammation-related disease is acute bronchial asthma.

5. The method of claim 2, wherein the inflammatory bowel disease is Crohn's disease or ulcerative colitis.

6. The method of claim 2, wherein the severity of symptoms associated with the eosinophilic lung inflammation-related disease or chronic obstructive pulmonary disease is quantitated by at least one of an increase in the CD4⁺ subset of T lymphocytes and a decrease in the CD8⁺ T lymphocytes, eosinophilic infiltration in lung tissue, increased levels of IL-5, or an increased CD11c+/CD11b+ DC/macrophage subset in bronchoalveolar lavage, compared to wild type mice at the RUNX3 locus.

7. The method of claim 2, wherein the symptoms of the inflammatory bowel disease comprise one or more of typhlocolitis, gastric mucosal hyperplasia, proliferative gastritis or proximal duodenitis.

8. The method of claim 2, which further comprises prior to the treatment, subjecting the mouse to environmental agents that induce exacerbation of the symptoms of the disease.

9. A method of predicting an increased risk for a chronic inflammatory disease in a subject which comprises: obtaining a test sample from the subject to be assessed; and determining expression of RUNX3 in the sample, wherein when the expression of RUNX3 in the test sample is diminished compared to normal levels expressed in healthy subjects, the subject is predicted to have an increased risk of susceptibility to a chronic inflammatory disease.

10. The method of claim 9, wherein the chronic inflammatory disease is an eosinophilic lung inflammation-related disease, a chronic obstructive pulmonary disease or an inflammatory bowel disease.

11. The method of claim 9, wherein the expression of RUNX3 is determined by measuring a level of mRNA specific for the RUNX3 gene or a level of RUNX3 protein.

12. The method of claim 9, wherein the test sample is obtained from peripheral blood mononuclear cells (PBMC).

13. The method of claim 10, wherein the subject is a human subject and wherein the chronic obstructive pulmonary disease is chronic bronchitis, the eosinophilic lung inflammation-related disease is acute bronchial asthma, or the inflammatory bowel disease is Crohn's disease or ulcerative colitis.

14. The method of claim 11 wherein the level of mRNA specific for the RUNX3 gene is measured by Northern blot, in situ hybridization or RT-PCR.

15. The method of claim 12 wherein the level of the Runx3 protein is measured by ELISA, radioimmunoassay, western blot or immunohistochemistry.

16. A method of testing the efficacy of a treatment for a chronic inflammatory disease comprising subjecting cells derived from a knockout mouse that is homozygous for a RUNX3 null allele to a putative treatment in vitro and determining the efficacy of the treatment.

17. The method of claim 16, wherein the chronic inflammatory disease is selected from: an eosinophilic lung inflammation-related disease, a chronic obstructive pulmonary disease and an inflammatory bowel disease.

18. The method of claim 16, wherein the cells are DC and wherein the efficacy of the treatment is determined by measuring the proportion of mature DC versus immature DC.

19. The method of claim 18 wherein the efficacy of the treatment is determined by measuring the proportion of DC expressing at least one of CD80, CD86, MHC class 11 and OX40L, wherein the treatment is effective against the chronic inflammatory disease if there is a reduction in the proportion of DC expressing at least one of CD80, CD86, MHC class 11 and OX40L.

20. A kit for diagnosis of genetic susceptibility to a chronic inflammatory disease comprising at least one probe capable of determining at least one genotype associated with the RUNX3 gene, or the expression of the gene product encoded by this locus, wherein the probe is adapted for determining at least one SNP associated with the RUNX3 gene.
